(19) United States
(12) Patent Application Publication
(10) Pub. No.: US 2021/0162067 A1
(43) Pub. Date: Jun. 3, 2021

(54) Composition and Methods of Controllable Co-Coupling Polypeptide Nanoparticle Delivery System for Nucleic Acid Therapeutics

(71) Applicant: SIRNAOMICS, INC., Gaithersburg, MD (US)

(72) Inventors: Xiaoyong LU, Gaithersburg (CN); Patrick Y. LU, Gaithersburg, MD (US); Vera SIMONENKO, Gaithersburg, MD (US); David M. EVANS, Gaithersburg, MD (US)

(21) Appl. No.: 17/103,386

(22) Filed: Nov. 24, 2020

Related U.S. Application Data
(63) Continuation of application No. PCT/US2019/033829, filed on May 23, 2019.
(60) Provisional application No. 62/676,218, filed on May 24, 2018.

(51) Int. Cl. A61K 47/69; C07K 4/00; A61K 48/00; C12N 15/113

(52) U.S. Cl. CPC A61K 47/6929; C07K 4/00; C12N 2310/14; C12N 15/113; A61K 48/005

(57) ABSTRACT

The present invention provides certain peptides and polypeptides useful in the preparation of nanoparticles for delivering nucleic acids and pharmaceutical drugs to mammalian cells and to humans and other mammals. It further provides methods for making the peptides, polypeptides, and nanoparticles and methods for using the nanoparticles.

### SEQUENCE LISTING

The instant application contains a Sequence Listing which has been submitted electronically in ASCII format and is hereby incorporated by reference in its entirety. Said ASCII copy, created on Feb. 17, 2021, is named 4690_0023C_SL.txt and is 31,228 bytes in size.

### CROSS-REFERENCE TO RELATED PATENT APPLICATIONS

This application is a continuation of International Application No. PCT/US2019/033829, filed on May 23, 2019, which claims the benefit of and priority to U.S. Provisional Patent Application No. 62/676,218, filed May 24, 2018, which is incorporated herein by reference in its entirety.

### FIELD OF INVENTION

The invention relates to certain peptides and polypeptides useful in the preparation of nanoparticles for delivering nucleic acids and pharmaceutical drugs to mammalian cells and to humans and other mammals.

### BACKGROUND OF THE INVENTION

Among the potential novel biologic drugs, including nucleotide-based medicines, such as microRNA (miRNA), small interfering RNA (siRNA), and DNA vaccines, the potential of RNAi to silence any gene has made it an attractive therapeutic modality, since the discovery of a functional RNAi pathway in mammals has provided a powerful tool for reverse genetics as a method for identifying gene function. Recently, siRNA has become a promising novel therapeutic candidate for treating many diseases, such as cancer, infections, macular degeneration, cardiovascular disease, nervous system disorders, and other gene-related diseases because of its sequence-specific post-transcriptional gene silencing ability. Due to their ability to reduce expression of any gene, siRNAs have been heralded as ideal candidates for treating a wide variety of diseases including “undruggable” targets.

However, the main challenge limiting RNAi as a potential clinical drug is the need for an effective delivery vehicle. An effective delivery vehicle must protect and transport its payload and, upon encountering cells, must cross the plasma membrane and gain access to the cytosolic compartment, where the RNAi machinery is located. Significant barriers to delivering siRNA into the cytoplasm include: (a) live cells have a very low permeability to high molecular weight molecules, such as proteins and oligonucleotides, (b) cell membranes typically have an overall negatively charged double layer structure, so it is very difficult for the negatively charged siRNA to permeate and cross over the membrane to enter the cell; (c) siRNA has a low stability and thus it is degraded in a very short period of time by various enzymes existing in plasma at high concentrations in vivo; (d) endosomal escape of the transported siRNA delivery complex to translocate into the cytosol and reach its target gene is another important consideration; and (e) siRNA may be recognized as a foreign substance and induce adverse immune effects. An ideal delivery system should address a majority of these technical challenges in order to achieve the desired therapeutic benefits.

Recently, lipid nanoparticles (LNPs), containing ionizable cationic lipids, such as 1,2-dilinoleyloxy-3-dimethylaminopropane (DLinDMA) have been used to deliver siRNA to the liver. More than 20 clinical studies are currently under way to evaluate the clinical applications of siRNAs. Examples of local delivery of siRNA include: through an ocular route for age-related macular degeneration [AMD] (Quark Pharmaceuticals, proangiogenic factor, Phase II); epidermal route for pachyonychia congenita [PC] (TransDerm; keratin 6a gene, Phase Ib); pulmonary route for asthmatic symptoms (ZaBeCor Pharmaceuticals; kinase Syk, Phase II); nasal route for respiratory syncytial virus [RSV] infection (Alnylam Pharmaceuticals; RSV nucleocapsid protein, Phase II); and oral route for familial adenomatous polyposis [FAP] (Marina Biotech, β-catenin, Phase I/II). Examples of systemic delivery of siRNA include; using cationic lipid nanoparticles stable nucleic acid lipid particle (SNALP)[1,2] for solid tumors (Tekmira Pharmaceuticals; polo-like kinase 1 [PLK1], Phase I) and hepatocyte carcinoma (Alnylam Pharmaceuticals; and vascular endothelial growth factor [VEGF] and kinesin spindle protein [KSP], Phase I) [3]. Moreover, Arrowhead Research (Calando Pharmaceuticals) has developed a dynamic polyconjugated delivery system (DPC) using cholesterol-conjugated siRNAs for hepatitis B virus (HBV) infection (Phase I clinical trial) [4]. In this delivery system, the siRNA is conjugated to an amphipathic poly(vinyl ether) (PBAVE). through a reversible disulfide linkage together with polyethylene glycol (PEG) and hepatocyte targeting ligand of N-acetylgalactosamine. Nanoparticle delivery systems have a pronounced advantage over the other methods. [5] Specifically, lipid nanoparticle (LNPs) have become one of the most advanced delivery platforms in systemic delivery of siRNA among other newly emerging delivery platforms. [6]

Recently, Sirnaomics Inc. (Gaithersburg, Md.) developed a histidine-lysine rich polypeptide delivery system for systemic delivery of dual siRNA (transforming growth factor-beta, TGF-β1, and cyclooxygenase-2, COX-2) to achieve a synergistic effect for hypertrophic scar reduction and prevention (Phase II, clinical trial) and treatment of liver fibrosis disease or other fibrosis diseases[7,8]. In this delivery system, the stable nanoparticle was formed between a positively charged polypeptide and a negatively charged siRNA, mainly through electrostatic interaction and hydrogen bonding. It has demonstrated good safety and efficacy in the current clinical trials, and it represents a novel class of delivery systems for delivering multi sequence-specific targeting siRNAs to achieve the dual therapeutic purpose to treat various diseases. [9]

The present invention includes a biodegradable polypeptide (referred to as ‘HKC2-nucleic acid delivery system’) in which a biocompatible polypeptide is complexed with nucleic acids through favored noncovalent interactions to form nanoparticles. The polypeptide is self covalently cross-linked through a biodegradable covalent bond in a histidine-lysine rich peptide in biocompatible conditions. This overall design and delivery system increase the in vivo stability and delivery efficiency of nucleic acids and can be used as an effective means for obtaining silencing in specific tissues. The HKC2-nucleic acid delivery system is a novel nanoparticle delivery carrier applicable to various disease treatments, functioning by complexing nucleic acids with a HKC2 peptide alone or in the presence of a co-delivery agent consisting of a branched polypeptide (HKP). This peptide has an appropriate positive charge and has a functional group which can be further modified for targeting specificity and reducing toxicity.

### BRIEF DESCRIPTION OF THE FIGURES

FIG. 1. Polypeptide nanoparticle formation between (A) in situ cross-linked cell penetrating peptide (CPP) HKC peptide with a specific peptide sequence, such as K(HHHK)4C-X-C (SEQ ID NO: 1), or KHHHKHHHHKHHHKHHHKC-X-C (SEQ ID NO: 2) and (B) already cross-linked polypeptide and selected siRNA, and its intracellular delivery and the intracellular release mechanism of HKC polypeptide-siRNA nanoparticle upon exposure to the intracellular reductive chemical GSH and its enzymatic production. X is a linker within the peptide sequence or could be a short chemical linker. Figure also discloses “GSSG” as SEQ ID NO: 58.

FIG. 2A. Structure of a) HKP (H3K4b) and HKP(+H) branched peptide, b) structure of the H3K4C2 (abbreviated as HKC2) with two cysteines located at the terminal site (SEQ ID NO: 3), and c) HKC general structure (SEQ ID NO: 59).

FIG. 2B. The HPLC chromatogram and integration table of HKC2, run on a C18 reversed phase HPLC column, with the peak eluting at a retention time of 8.053, or >91% of the gradient produced between water (0.065% TFA) and acetonitrile (0.05% TFA).

FIG. 2C. Mass spectroscopy (ESI-MS, positive) of the HKC2, demonstrating an observed double charged molecular ion peak at 1343 [M]²⁺.

FIG. 3. Figure showing the mechanism of HKC polypeptide formation through cross linking induced by oxidation using oxygen or DMSO and degradation under reduction by glutathione.

FIG. 4. Figure showing the design and post targeting ligand functionalization of the HKC2 through a thiol-maleimide reaction on the free thiol exposed on the surface of a polypeptide nanoparticle PNP which can be complexed with siRNA allowing targeted delivery of the product to cells with specific receptors. Upon entry, intracellular cleavage of S—S bond by GSH (glutathione) releases the siRNA, allowing silencing of the gene targeted by the siRNA.

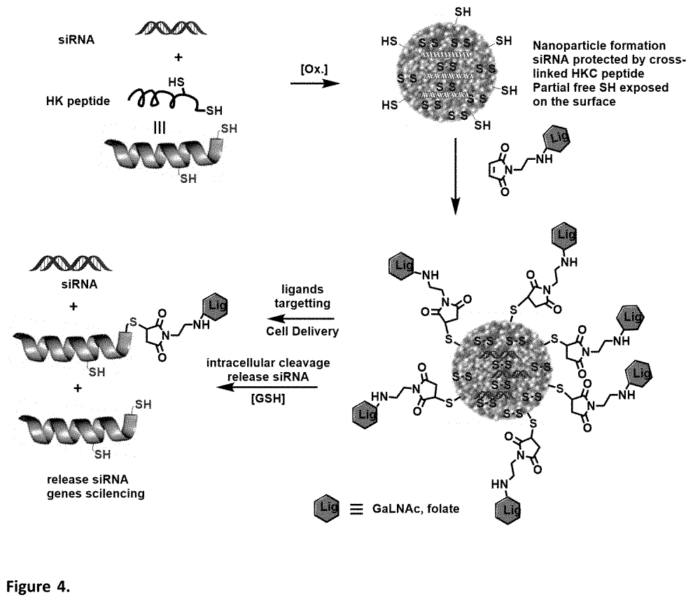

FIG. 5. The size distribution of polynanoparticles formed between HKC2 and TGFβ1 measured using Dynamic Light Scattering instrument (DLS). HKC-siRNA particles were measured for size using a 90plus Nanoparticle Size Distribution Analyser (Brookheaven Instruments Limited, NY). Solution of TGFβ1 (25 ng/μL in water) was added to HKC2 (300 ng/μL in water) and mixed at room temperature. The resulting mixture was stirred vigorously and stored for 30 min before DLS (Dynamic Light Scattering) measurement. DLS was measured by dilution of the mixture to the 2.0 mL volume of the cuvette. The result indicated that the average size of this preparation of HKC-siRNA nanoparticle ranged between 206 nm to 64 nm as the ratio of HKC2 to siRNA was increased. The Zeta-potential value was +10.

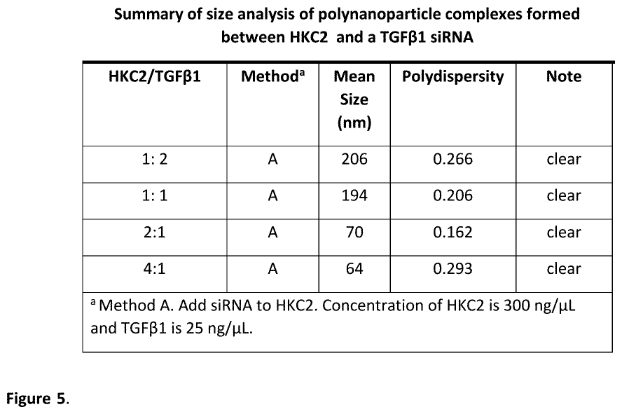

FIG. 6. The size distribution of polynanoparticle between HKC2 and TGFβ1 siRNA measured using DLS. An aqueous solution of TGFβ1 siRNA (25 ng/μL) was added to an aqueous solution of HKC2 (25 ng/μL) and mixed at room temperature. The resultant mixture was stirred vigorously and incubated at RT for 30 min before DLS measurement. DLS was measured after dilution of the resultant mixture in a 2.0 mL-volume cuvette.

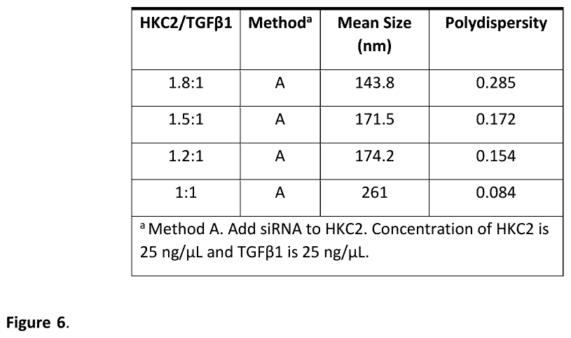

FIG. 7. Evaluation of HKC2 peptide as an siRNA carrier. HEK293 cells were seeded at 3×10⁴ cells per well in a 48-well plate and incubated overnight. On the next day AF488-labeled siRNA/HKC2 complexes were prepared as follows: an aqueous solution of siRNA (0.025 μg/μL, 21-mer) a HKC2 (0.05 μg/μL) were combined at following HKC2 to siRNA mass ratios: 1:1, 1.7:1, 2:1, 4:1, 8:1 and 1:2. In 30 min siRNA/HKC2 complexes were added to the cells. Fluorescent images were taken 24 h after transfection.

FIG. 8. HKC2 peptide-mediated delivery of fluorescently labeled siRNA (Alexa Fluor 488) into A549 cells. A549 cells were seeded in the wells of a 48-well plate at a density 3×10⁴ cells/well on the day before transfection. On the next day AF488-labeled siRNA/HKC2 complexes were prepared as follows: an aqueous solutions of siRNA (25 ng/μL, 21-mer) and HKC2 (50 ng/μL) were combined at following HKC2 to siRNA ratios: 1:1, 1.7:1, 2:1, 4:1, 8:1 and 1:2. In 30 min siRNA/transfection reagent complexes were added to the cells. Fluorescent images were taken 24 h after transfection.

FIG. 9. Gel retardation assay to determine the amount of HKC2 that retards siRNA migration. Various ratios of HKC2 in complex with siRNA (TGFβ1, 500 ng) were prepared and subjected to gel electrophoresis for 30 min (3% gel). Different ratios of HKC polypeptide to siRNA were represented above the gel. In practice, 25 ng/μL of siRNA was incubated with various amounts of HKC2 peptide in ratios of 1:2, 1:1, 1.5:1, 2:1, 3:1, 4:1. and reference HKP (4:1). Following an incubation for 20 min, 20 uL of siRNA/peptide (500 ng siRNA in each) complex was loaded in the wells. The free and bound siRNA was separated on a 3.0% non-denaturing agarose gel under 100V applied for 30 min. The gel was prestained with Ethidium bromide RNA dye, and the resulting fluorescent bands UV=290 nm were visualized with a Fuji LAS4000 Imager. The results presented are representative of the images observed.

FIG. 10. Gel retardation assay to validate that degradable HKC can release siRNA in the presence of glutathione (GSH). Various ratios of HKC2 or HKP in complex with siRNA (TGFβ1, 500 ng) were prepared and subjected to gel electrophoresis for 30 min (3% gel). Different ratios of HKC2 polypeptide to siRNA are shown (above the gel). In practice, 25 ng/μL of siRNA was incubated with various amounts of cross linked HKC2 peptide in ratio of 4:1 and 8:1. Reference HKP (4:1) or the product were incubated in the presence or absence of 20 mM glutathione (GSH). Following the incubation for 40 min, 20 uL of siRNA/peptide (500 ng siRNA in each) complex was loaded into the wells of the gel. The free and bound siRNA were separated on a 3.0% agarose gel under 100V applied voltage for 30 min. The gel was stained with Ethidium bromide RNA dye, and the resulting fluorescent bands (UV=290 nm) were visualized with a Fuji LAS4000 Imager. The results presented are representative of the images obtained.

FIG. 11. Size distribution of formulation of HKC2:HKP:TGFβ1 in the formation of nanoparticle. HKC2=K(HHHK)₄CSSC (SEQ ID NO: 3). HKP=H3K4b. TGFβ1 was used in 80 ng/μL in water. They were mixed with an equal volume of the HKC and HKP in water. The nanoparticle formation of HKC2, HKP and siRNA (TGFβ1) was evaluated at various ratios of each. The addition of the HKC2 into the HKP/siRNA formulation maintained a similar nanoparticle size but significantly reduced the range of sizes as shown by the reduction in polydispersity index (PDI) compared to the control HKP/siRNA (N:P mass ratio=4:1). The HKC2/HKP/siRNA was formulated in mass ratio 0:4:1, 1:4:1, 1:3:1, 2:3:1, 2:2:1, 3:1:1. An aqueous solution of HKC2 (160 ng/μL), HKP (320 ng/μL) and siRNA (80 ng/μL) was mixed in the defined ratio and incubated at RT for 30 min. The resultant sample was subsequently measured by dynamic light scattering using a Nanoplus 90 instrument. The dynamic radius was recorded and shown in FIG. 11.

FIG. 12. Polydispersity of HKC2:HKP:TGFβ1 in the formation of nanoparticles. HKC2=K(HHHK)₄CSSC (SEQ ID NO: 3). HKP=H3K4b. TGFβ1 was used in 80 ng/μL in water. They were mixed with equal volume of the HKC and HKP in water. The nanoparticle formation of HKC2, HKP and siRNA (TGFβ1) was evaluated at various ratios. The addition of the HKC2 into the HKP/siRNA formulation maintained a similar nanoparticle size but significantly narrowed the polydispersity index (PDI) compared with the control HKP/siRNA (N:P mass ratio=4:1). The HKC2/HKP/siRNA was formulated in mass ratios of 0:4:1, 1:4:1, 1:3:1, 2:3:1, 2:2:1, 3:1:1. An aqueous solution of HKC2 (160 ng/μL), HKP (320 ng/μL) and siRNA (80 ng/μL) was mixed in the defined ratio and incubated at RT for 30 min. The resultant sample was subsequently measured by dynamic light scattering using a Nanoplus 90. The dynamic radius was recorded and shown in FIG. 12.

FIG. 13. Effect of treatment with CellDeath siRNA formulated with HKP alone or in combination with various amount of HKP and HKC on human glioblastoma T98G cell line. Various mass ratios of HKP/HKC2/siRNA were used and lipofectamine was also used for a control. At first an aqueous solution of HKC (160 ng/ul) was added to an aqueous solution of siRNA (80 ng/ul), mixed, briefly vortexed, then in the same manner was added HKP (320 ng/ul). Mixtures were incubated at RT for 30 min. Transfection complexes were diluted with OPTI-MEM and added to the cells in 100 ul medium supplied with fresh medium. After a 6 h incubation, transfection medium was replaced with 10% FBS/DMEM or EMEM. At 72 h post-transfection the number of viable cells was assessed with CellTiter-Glo Luminescent cell viability assay (Promega). Values derived from untreated cells (Blank) were set as 100%. All values represent the mean of ±S.D. of four replicates NS-non-silencing siRNA, CD-CellDeath siRNA.

FIG. 14. Effect of treatment with CellDeath siRNA formulated with HKP alone or in combination with various amounts of HKP and HKC on human hepatocellular carcinoma HepG2 cells. Various mass ratios of HKP/HKC2/siRNA were used and lipofectamine was also used as a control. At first an aqueous solution of HKC2 (160 ng/ul) was added to an aqueous solution of siRNA (80 ng/μL), mixed, briefly vortexed, then in the same manner HKP (320 ng/μL) was added. Mixtures were incubated at RT for 30 min. Transfection complexes were diluted with OPTI-MEM and added to the cells in 1004 medium supplemented with fresh medium. 6 h after transfection, medium was replaced with 10% FBS/DMEM or EMEM. At 72 h post-transfection the number of viable cells was assessed with CellTiter-Glo Luminescent cell viability assay (Promega). Values derived from untreated cells (Blank) were set as 100%. All values represent the mean of ±S.D. of four replicates NS=non-silencing siRNA, CD=Cell Death siRNA (siRNA that kills cells when it is introduced by transfection).

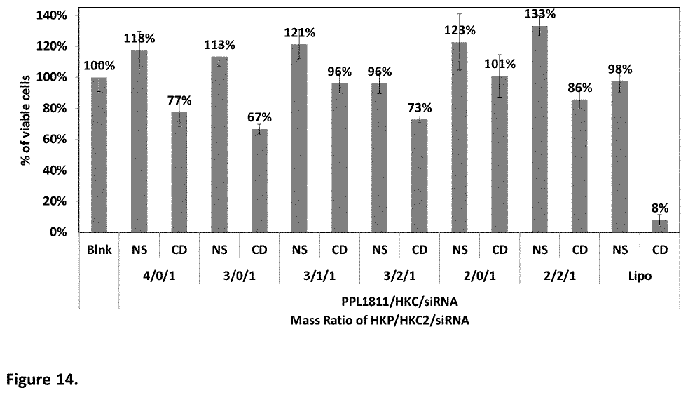

### DESCRIPTION OF THE INVENTION

The current invention provides certain peptides and polypeptides useful in the preparation of nanoparticles for delivering nucleic acids and pharmaceutical drugs to mammalian cells and to humans and other mammals.

### Peptides

The invention includes a peptide with the formula Kp{[(H)n(K)m]}y-C-x-Z (SEQ ID NO: 4) or with the formula Kp{[(H)a(K)m(H)b(K)m (H)c(K)m(H)d(K)m]}y-C-x-Z (SEQ ID NO: 5), where K is lysine, H is histidine, C is cysteine, x is a linker, Z is a mammalian cell-targeting ligand, p is 0 or 1, n is an integer from 1 to 5 (preferably 3), m is an integer from 0 to 3 (preferably 0 or 1), a, b, c, and d are either 3 or 4, and y is an integer from 3 to 10 (preferably 4 or 8). In one embodiment, the peptide has the formula K[(H)n(K)m]y-C-x-C (SEQ ID NO: 6), where K is lysine, H is histidine, C is cysteine, n is an integer from 1 to 5 (preferably 3), m is an integer from 0 to 3 (preferably 0 or 1), y is an integer from 3 to 7 (preferably 4), and x is a linker. The peptides may be linear or branched. They are capable of being internalized into a mammalian cell, preferably a human cell, such as a human tumor cell.

The mammalian cell-targeting ligand (Z) is a peptide, a protein, an antibody, a small molecule, a carbohydrate moiety, or an oligonucleotide. The targeting ligand is a molecule that will bind to a specific receptor on the specific cell surface and internalize its payload thereafter.

In one embodiment, Z is a peptide 1-60 amino acids in length. In one aspect of this embodiment, Z is one amino acid, preferably C. In another aspect, if Z is more than 1 amino acid, it may include a ‘spacer region’ of several inert amino acids (e.g. serines). Z may further include a peptide ligand that targets a receptor on the surface of mammalian cells (e.g. the transferrin receptor, EGFR, or GLP1R). There are many examples of receptors that are exclusively expressed on cell types of interest, and any ligand that can bind these receptors may help with specific localized delivery of the siRNA to the cells expressing this receptor.

In one embodiment, x is a single amino acid residue or a peptide sequence with 2-15 amino acids. In one aspect of this embodiment, the peptide sequence has 3-8 amino acids.

The invention also includes a peptide with the formula K[(H)n(K)m]y-C(SEQ ID NO: 7), where K is lysine, H is histidine, C is cysteine, n is an integer from 1 to 5 (preferably 3), m is an integer from 0 to 3 (preferably 0 or 1), y is an integer from 3 to 7 (preferably 4).

### Polypeptides

The invention includes a polypeptide comprising at least 2 of the peptides described above cross-linked through disulfide bonds. The polypeptide may be linear or branched. The bonds are biodegradable cysteine disulfide bonds. Alternatively, the biodegradable cysteine disulfide bond can be replaced by any cleavable bond including, but not limited to, anhydride bond, a hydrazine bond, an enzyme-specific peptide bond, or a combination thereof.

### Nanoparticles

The invention includes a nanoparticle comprising one or more of the previously described polypeptides and a nucleic acid. The nanoparticle may further include a histidine-lysine copolymer, a second nucleic acid, and/or a pharmaceutical drug. The nanoparticle is capable of being internalized into a mammalian cell. In one embodiment, the polypeptide and the nanoparticle are biodegradable in a mammalian cell, such as by glutathione reduction or enzyme or pH change within the cell. In one aspect of these embodiments, the nanoparticle size is 50-300 nm. In another aspect, the nanoparticle size is 80-130 nm with a polydispersity index of 0.2 or below.

The nucleic acid or acids comprise an siRNA, an miRNA, an antisense oligo, a plasmid, an mRNA, an RNAzyme, a DNAzyme, or an aptamer sequence.

In one embodiment, the nucleic acid comprises an siRNA. As used herein, an “siRNA” or an “siRNA molecule” is a duplex oligonucleotide, that is a short, double-stranded polynucleotide, that interferes with the expression of a gene in a cell that produces RNA, after the molecule is introduced into the cell. For example, it targets and binds to a complementary nucleotide sequence in a single stranded (ss) target RNA molecule, such as an mRNA or a micro RNA (miRNA). The target RNA is then degraded by the cell. Such molecules are constructed by techniques known to those skilled in the art. Such techniques are described in U.S. Pat. Nos. 5,898,031, 6,107,094, 6,506,559, 7,056,704, RE46,873 E, and 9,642,873 B2 and in European Pat. Nos. 1214945 and 1230375, all of which are incorporated herein by reference in their entireties. By convention in the field, when an siRNA molecule is identified by a particular nucleotide sequence, the sequence refers to the sense strand of the duplex molecule.

The siRNA molecule can be made of naturally occurring ribonucleotides, i.e., those found in living cells, or one or more of its nucleotides can be chemically modified by techniques known in the art. In addition to being modified at the level of one or more of its individual nucleotides, the backbone of the oligonucleotide can be modified. Additional modifications include the use of small molecules (e.g. sugar molecules), amino acid molecules, peptides, cholesterol, and other large molecules for conjugation onto the siRNA molecule.

In one embodiment, the molecule is a double-stranded oligonucleotide with a length of 16-27 base pairs. In one aspect of this embodiment, the molecule is an oligonucleotide with a length of about 19 to about 27 base pairs. In another aspect, the molecule is an oligonucleotide with a length of about 21 to about 25 base pairs. In all of these aspects, the molecule may have blunt ends at both ends, or sticky ends at both ends, or a blunt end at one end and a sticky end at the other. In one aspect, the sticky ends have overhangs of 1-3 nucleotides. In another aspect of this embodiment, the nucleic acid comprises an siRNA molecule identified in Tables 1-3 herein.

The siRNA molecules of the invention include molecules derived from those identified in Tables 1-3. These include: a) a derived duplex consisting of 24 contiguous base pairs of any one of the duplexes in Tables 1-3; b) a derived duplex consisting of 23 contiguous base pairs of any one of the duplexes in Tables 1-3; c) a derived duplex consisting of 22 contiguous base pairs of any one of the duplexes in Tables 1-3; d) a derived duplex consisting of 21 contiguous base pairs of any one the duplexes in Tables 1-3; e) a derived duplex consisting of 20 contiguous base pairs of any one of the duplexes in Tables 1-3; f) a derived duplex consisting of 19 contiguous base pairs of any one of the duplexes in Tables 1-3; g) a derived duplex consisting of 18 contiguous base pairs of any one of the duplexes in Tables 1-3; h) a derived duplex consisting of 17 contiguous base pairs of any one of the duplexes in Tables 1-3; and i) a derived duplex consisting of 16 contiguous base pairs of any one of the duplexes in Tables 1-3.

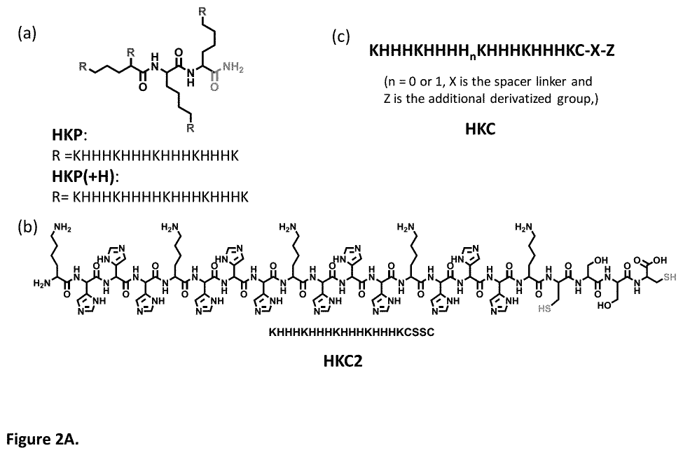

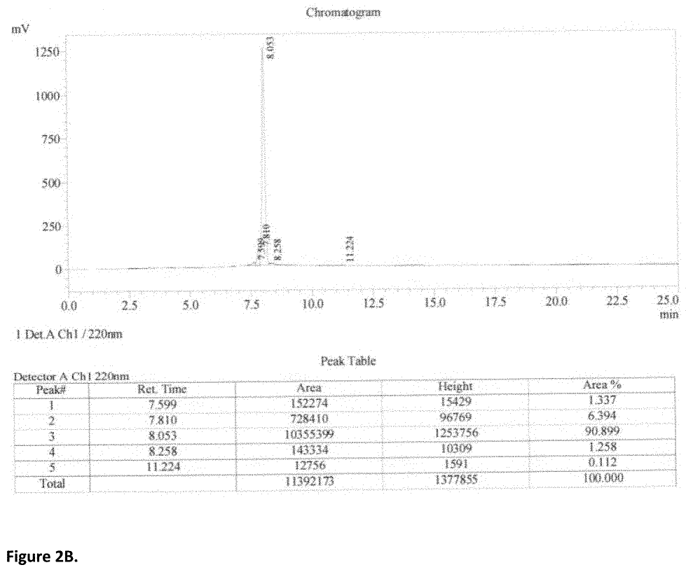

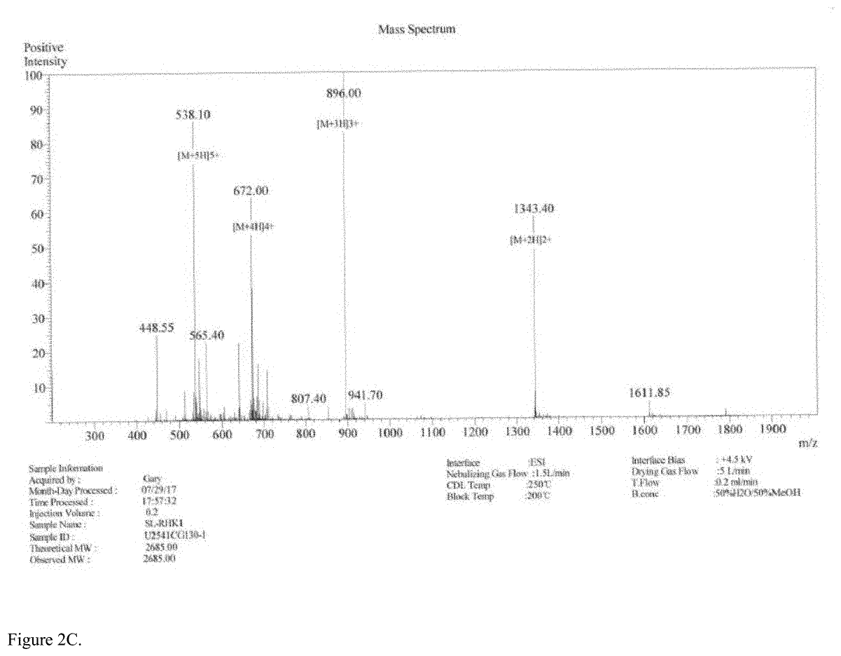

The histidine-lysine copolymer (HKP) is disclosed in U.S. Pat. No. 7,070,807 B2, issued Jul. 4, 2006, U.S. Pat. No. 7,163,695 B2, issued Jan. 16, 2007, U.S. Pat. No. 7,772,201 B2, issued Aug. 10, 2010, RE46,873 E, issued May 29, 2018, and U.S. Pat. No. 9,642,873 B2, issued May 9, 2017 all of which are incorporated by reference herein in their entirety. In one embodiment, this copolymer comprises H3K4b. In another embodiment, it comprises HKP(+H). See FIG. 2A.

In one embodiment, the nanoparticle further includes a functional group attached through a partially free thiol group residue. In one aspect of this embodiment, the thiol group residue is on the nanoparticle's surface. It is added after the nanoparticle's formation. In another aspect, the thiol group residue is on a cytosine sidechain within a peptide sequence. It is added before the nanoparticle's formation.

The functional group is selected from the group consisting of a small molecule (e.g., a molecule that can bind to cell surface receptors or a molecule that can induce cell killing when internalized, such as doxorubicin or gemcitabine), a protecting polyethylene glycol (PEG) molecule, a lipid, a peptide or protein (e.g., an antibody), or an oligonucleotide (e.g., an aptamer or 1 strand of an siRNA molecule), and an organic molecule with carbohydrate binding sites that recognize asialoglycoprotein receptors (ASGPRs) (e.g., GalNac, Mannose 6P, asialofetuin, etc.). The peptide/protein/carbohydrate sugar groups and other entities have affinity for receptors present on discrete cells and allow binding of the nanoparticles to these cells with uptake of the nanoparticles into the cells. For example, GalNac binds to ASGPRs on hepatocytes and has shown specificity for hepatocytes within the liver. In one particular aspect, the functional group is a protecting PEG molecule to assist with improved biodistribution or minimize non-specific binding to cells.

In a further embodiment, the nanoparticle includes a pharmaceutical drug. In one aspect of this embodiment, the drug is selected from the group consisting of a small molecule drug, a peptide drug, and a protein drug.

### Methods of Making

The peptides and polypeptides of the invention are prepared by techniques known to those skilled in the art in view of the teachings disclosed herein. In one embodiment, the peptides are prepared by a method comprising the steps of: a) linking the initial lysine (K) to a solid support; b) linking additional amino acids one after another to the initial lysine; and c) recovering the synthesized peptide. In one embodiment, the polypeptides are prepared by a method comprising the steps of: a) cross-linking the peptides of the invention by chemical oxidation to form a polypeptide with cleavable bonds, and b) recovering the polypeptide. In one aspect of this embodiment, the cleavable bonds are disulfide bonds.

The nanoparticles of the invention are prepared by techniques known to those skilled in the art in view of the teachings disclosed herein. In one embodiment, the nanoparticles are prepared by a method comprising the steps of: a) cross-linking the peptides of the invention by chemical oxidation to form polypeptides with cleavable bonds, b) mixing the polypeptides with a nucleic acid, and c) recovering the nanoparticles. In one aspect of this embodiment, the cleavable bonds are disulfide bonds. In another embodiment, the nanoparticles are prepared by a method comprising the steps of: a) mixing the polypeptides of the invention with a nucleic acid to form a nanoparticle, and b) recovering the nanoparticle. In still another embodiment, the nanoparticles are prepared by a method comprising the steps of: a) mixing the peptides of the invention with a nucleic acid, b) cross-linking the peptides by chemical oxidation to form a polypeptide with cleavable bonds, resulting in the formation of a nanoparticle, and c) recovering the nanoparticle. In one aspect of this embodiment, the cleavable bonds are disulfide bonds. In one aspect of these embodiments, the polypeptide and the nucleic acid are mixed in an aqueous solution, such as an aqueous buffer with a pH range of 6.0-8.0. In a further aspect of these embodiments, the nanoparticle is formulated with a nitrogen to phosphate (N:P) ratio (w:w=2:1-8:1) in a range of controllable mixing conditions. In a still further aspect of these embodiments, the nucleic acid is an siRNA, an miRNA, an antisense oligo, a plasmid, an mRNA, an RNAzyme, a DNAzyme, or an aptamer sequence.

In one embodiment, the method of making the nanoparticles of the invention includes the additional step of adding a histidine-lysine copolymer. The percentage of the histidine-lysine copolymer ranges from 20% to 97%.

In another embodiment, the method of making the nanoparticles of the invention includes the additional step of mixing a pharmaceutical drug with the polypeptide and the nucleic acid. The pharmaceutical drug comprises a small molecule drug, a peptide drug, or a protein drug.

### Methods of Use

The nanoparticles of the invention are useful for delivering nucleic acids and pharmaceutical drugs to humans, other mammals, and mammalian cells.

The invention includes a method of delivering a nucleic acid to a mammalian cell comprising delivering a sufficient amount the nanoparticles of the invention to the cell under conditions wherein the nanoparticles are taken into the cell and release the nucleic acid. As previously described, the nucleic acid comprises an siRNA, an miRNA, an antisense oligo, a plasmid, an mRNA, an RNAzyme, a DNAzyme, or an aptamer sequence. In one aspect, the nucleic acid is delivered to the cell in vitro. In another aspect, it is delivered to the cell in vivo. In one aspect, the mammalian cell is the cell of a laboratory animal. Such laboratory animals include rodents, dogs, cats, and nonhuman primates. In another aspect, the mammalian cell is a human cell. In one particular aspect, the nucleic acid is an siRNA, examples of which are described above.

The invention further includes a method of gene therapy in a mammal comprising administering a therapeutically effective amount of the nanoparticles of the invention to the mammal. A sufficient amount of the nanoparticles is delivered to the mammal under conditions where the nanoparticles are taken up by the target cells and the nucleic acid is released into the cells. In one embodiment, the mammal is a human. In another embodiment, the mammal is a laboratory animal, such as those identified in the preceding paragraph. In one aspect of these embodiments, the nucleic acid is an siRNA, examples of which are described above.

The invention further includes a method of delivering a therapeutic compound to a mammal comprising delivering a therapeutically effective amount of the nanoparticles of the invention to the mammal. A sufficient amount of the nanoparticles is delivered to the mammal under conditions where the nanoparticles are taken up by the target cells and the therapeutic compound is released into the cells. In one aspect, the mammal is a human. In another aspect, the mammal is a laboratory animal, such as those identified above.

The dosages, methods of administration, and times of administration are readily determinable by a person skilled in the art, given the teachings contained herein. In one embodiment, the composition is administered by injection into the tissue of the mammal. In another embodiment, the composition is administered by subcutaneous injection into the mammal. In still another embodiment, the composition is administered intravenously to the mammal. In a preferred embodiment, the mammal is a human.

### Experimental Design and Techniques

### Background

The current invention provides a nucleic acid delivery system. The system comprises a reduction-sensitive disulfide bond-bridged shielding system, which can include a targeting function, a positive charged polypeptide material, and a nucleic acid. These form a nanoparticle complex through noncovalent interaction between the positively charged peptide and negatively charged siRNA, where the surface is shielded by the polypeptide and toxicity is reduced. The stable complex delivers and transports the loaded genetic material into cells. In the reductive enriched intracellular environment (compared to the extracellular milieu), the delivery polypeptide is degraded by glutathione (GSH) and releases its payload nucleic acid sequence and completes the transfection process. Moreover, the advantage of the delivery system is its simplicity and effectiveness; the partially free cysteines on the surface of the nanoparticle allows for further coupling of a targeting ligand function. Such a targeting function can enhance the efficiency of the nucleic acid transfection into cells specifically targeted by the attached ligand.

The invention provides a polypeptide nanoparticle which comprises a cysteine-containing histidine-lysine rich peptide cross-linked through disulfide bonds and complexed with siRNA mainly through electrostatic interactions and hydrogen bonds.

The invention also provides at least one nucleic acid (and also two different nucleic acids) and a pharmaceutically acceptable carrier. In an example with siRNA, one of the duplexes binds to an mRNA molecule that encodes VEGF, and the other binds to an mRNA molecule that encodes VEGFR2. In one embodiment, the composition further comprises a siRNA duplex that binds to an mRNA molecule that encodes TGFβ1. In one aspect of these embodiments, the duplexes target both human mRNA and the homologous mouse mRNA.

The invention further relates to a redox active component, which could be a peptide or linear molecule, which can be cross-linked under oxidation conditions to form a polypeptide. The polypeptide is complexed with nucleic acid to form a nanoparticle. The size range is 50-300 nm, depending on the relative ratio between the two components. The size is preferably between 80-130 nm with a narrow polydispersity index value.

The invention further relates to a composition comprising a biodegradable peptide component and siRNA, mRNA, or DNA. It forms a nanoparticle or nanoaggregates. The complex formation effectively protects and delivers the siRNA, mRNA, or DNA into the cell. The siRNA or other cargos can be released in the reducing environment inside the cell (GSH concentration, 0.5-10 mM in the cytosol and 20 mM in the nucleus), which promotes the cleavage of the disulfide linkages, following higher uptake by target cells through endocytosis triggered by repeating histidine-lysine units.

### Design of Novel H3K4C2 System

This design is based on the success of previously established H3K4b [KKK(KHHHKHHHₙKHHHKHHHK)₄], HKP (where n=1), HKP(+H)(where n=2), see FIG. 2A] system for siRNA delivery in vitro and in vivo experiments. Two siRNAs (each targeting the same gene or different genes) was effectively complexed with H3K4b during the formulation to form a stable nanoparticle (^{˜}150 nm). It was intracellularly delivered upon binding to the cell, and then escaped from the endosome into the cytoplasm where the siRNA is able to effect gene silencing. After entrapped siRNA was released from the endosome, it induced gene silencing in the cancer cell. Despite its demonstration as a potent and effective carrier for delivery of dual targeting siRNA, there remains some room for improvement, including in the release of the negative charged siRNA from the tightly bound positively charged H3K4b nanoparticle. The binding could cause a decrease in the efficacy of the siRNA in the transfection step. In other words, a higher dosage of siRNA may have to be formulated to cause the therapeutic effect.

The biodegradable bond linkage in the polypeptide can be chosen from a disulfide bond, an anhydride bond, a hydrazine bond, an enzyme-specific cleavable peptide bond, and other chemistries known to one skilled in the art. Similarly, the bonds can be a combination of multiple bond types. Such a linkage can be degraded under a selective biological environment. In the current invention, the biodegradable bond (such as reduction sensitive S—S bond, low pH cleavable imine etc.) which connects the single peptide in the polypeptide to other moieties, may be biodegradable by a selected bio-stimulus, such as enzymatic exposure, change of pH e.g. increased acidity (pH control), and the specific biological environment (for example, in the presence of a high concentration of intracellular GSH in a tumor cell), or other chemical stimulus. Thus, the entrapped siRNA is released from the polypeptide nanoparticle of the HKC2 peptide due to the degradation of the polypeptide under the specific biological condition.

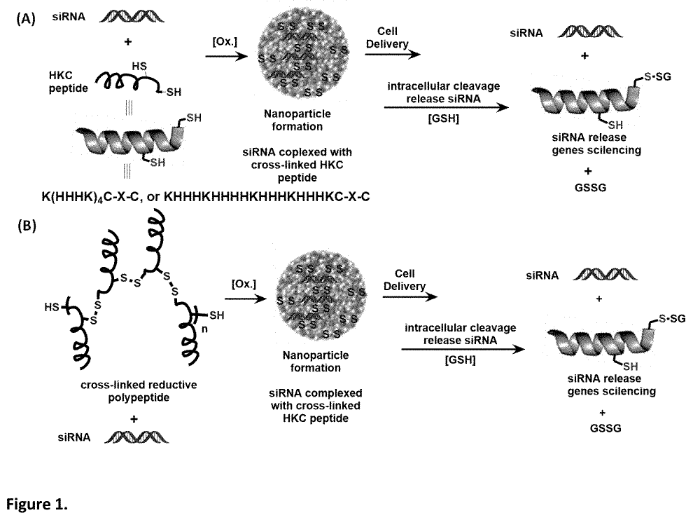

Subsequently, suppression of target gene expression by released siRNA will be achieved once it reaches its targeted gene. For example, in order to improve the release efficiency of siRNA to enhance efficacy of the siRNA delivered to the cell, a chemically biodegradable Histidine-Lysine-Cysteine HKC2 polymer was designed, based on the disulfide bond linkage between the cysteine in the branched HK and the cysteine in the backbone to form a polypeptide HKC2 with repeating units of a single branched HK, which have a similar structure to H3K4b. It results in effective protection of nucleic acid against nucleases, and stabilization during crossing the non-reductive environments, such as extracellular space and blood (glutathione [GSH] concentration, 0.5-10 μM). But this polymer, HKC2, can be cleaved when it is exposed to higher concentration of GSH (0.5^{˜}10 mM) inside of the cell. Particularly, considering the increased concentration of glutathione (GSH) in cancer cells in previous reports, the biodegradable linkage, such as the disulfide bond in the polypeptide-siRNA nanoparticle delivery carrier, can be effectively degraded to release and deliver the siRNA to its target.[10, 11] The cleavage of the S—S bonds that link the branched HK to the backbone will cut the branched HK into separate pieces, which one would expect to no longer form a stable complex with the siRNA. Therefore, the GSH will trigger the release of siRNA by degrading the HKC2 polymer/siRNA complex at the intracellular level (FIG. 1).

Design of a Redox Active HKC2s Polypeptide to Enhance the siRNA Release and Transfection Efficacy

### 1. Structure of Histidine-Lysine (HK) Branched Polymers

Among all of the Histidine Lysine (HK) Polymers that have been studied, including H2K, H3K, H3K4b (FIG. 2A), previous reports [12,13] and our formulation and efficacy studies have shown that H3K4b can form effective nanoparticles when complexed with siRNA. Based on reported experimental evidence, the linear structure of HK cannot effectively form a complex with and deliver siRNA. [12,13]. However, we also observed some of the slow release of siRNA during the transfection step based on the strong noncovalent interactions between the positive charged lysines in intact polymer H3K4b and the negatively charged phosphate backbone of the siRNA.

Therefore, there was a need to design and develop a more effective HKP polymer that can form a polymer with siRNA under oxidative conditions and break apart at the siRNA release step when the polymer is exposed to reductive conditions (such as high GSH concentration within tumor cells) [14,15]. Ideally, such a biodegradable responsive HKP polymer could effectively complex with siRNA to prevent its degradation during delivery, and finally release the entrapped siRNA efficiently in the cytoplasm to access the siRNA machinery to access therapeutic target mRNAs for silencing (FIG. 1).

### 2. Design and Preparation of a Biodegradable Histidine-Lysine-Cysteine HKC2 Polymer

Break down of the H3K4b polymer into four of the same linear peptide building blocks is shown below. Such a branched polymer can be prepared from two building blocks: a linear cysteine containing the peptide RSH and a multi free thiol containing backbone, through disulfide bond linkages. Such a S—S linkage is redox responsive. For example, SH can be oxidized into an S—S bond in the formulation with siRNA to form H3K4b polymer to entrap siRNA, but the S—S bond can be broken down when it is exposed to high concentrations of intracellular GSH and thus releases the siRNA. The peptide can be synthesized by continuous solid phase synthesis. We simplified the two chemical components into one peptide H3K42C bearing two cysteine sequences at the terminal site with a two amino acid spacing group (—CSSC (SEQ ID NO: 8), or any of C-linker-C type of sequence, Histidine-Lysine-Cysteine, abbreviated as HKC2) to reduce the possibility of disulfide bond cross linking within a molecule rather than between molecules. In this structure, the peptide has a lysine and three repeating histidine sequences (K(HHHK)₄CSSC (SEQ ID NO: 3)). This sequence has a similar structure as a single branch of polypeptide H3K4b. However, manufacturing this sequence can significantly reduce the synthesis cost compared with the branched polypeptide (FIG. 2A). SEQ ID NOS 9, 9, and 3 are disclosed below, respectively, in order of appearance.

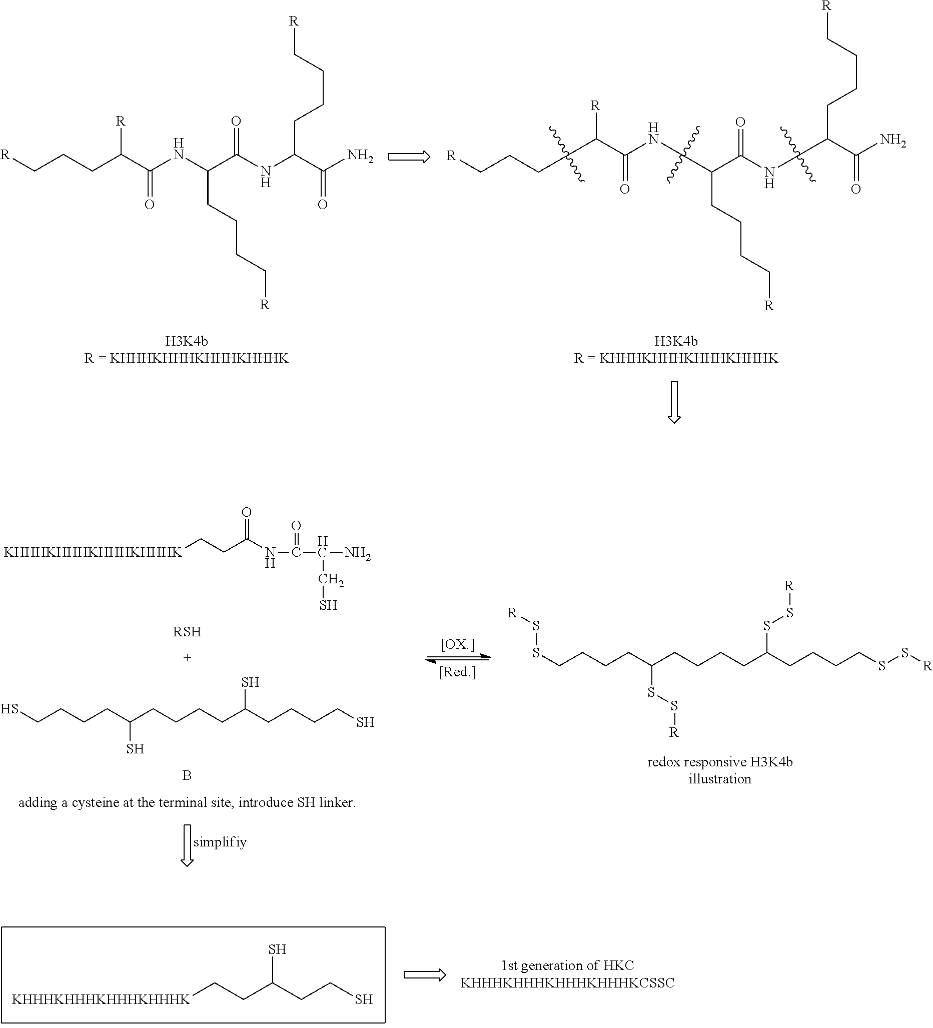

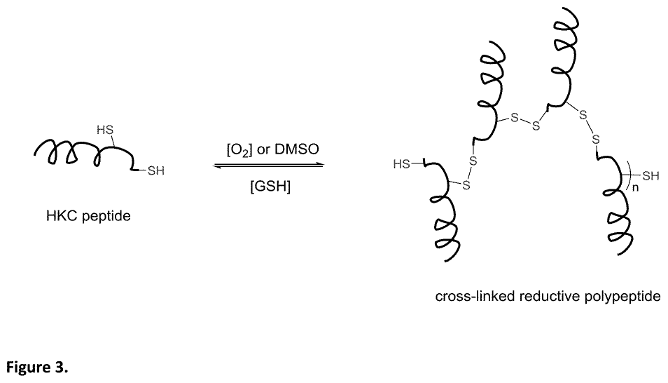

In the present invention, the biodegradable polypeptide-nucleic acid delivery system provides several advantages compared to other systems: 1.) The relative safety and efficacy of the similar polypeptide H3K4b has been investigated in various animal models and even in clinical trials. This biodegradable system would be more biocompatible than the synthetic polymer or a lipophilic system comprising mixed lipids. 2.) The relative low cost and ease of manufacture is a significant benefit during production. 3.) The polymer complex is biodegradable under physiological conditions. 4.) More than one nucleic acid can be loaded at the same time to achieve a synergistic therapeutic effect (targeting genes in multiple dependent or independent pathways). 5.) The polypeptide (cationic feature) and nucleic acid (negative charge surface) will bind together through the electrostatic interaction and hydrogen bonding interaction. 6.) The simplicity of the system will be another plus in practice. The self cross-linking is shown in FIG. 3 and FIG. 1.

The preparation of the polypeptide/nucleic acid delivery carrier described in the current invention by combining a polypeptide with a single or multiple nucleic acid(s) may be implemented by the following method, comprising the steps: (a) introducing biodegradable functional groups into a linear histidine-lysine rich peptide, such as two free thiol groups; (b) biologically covalently linking the peptides through disulfide bonds into a polypeptide through oxidation by air or using a low percentage of DMSO in aqueous media; (c) and combining the polypeptide made in step (b) with one or more siRNA molecules, mainly through favored charge interaction, to produce the stable nanoparticle.

Alternatively, the polypeptide/nucleic acid can also be produced by mixing the linear peptide and nucleic acid together. The polypeptide will be cross-linked in situ to provide the nanoparticle.

According to the mechanism of siRNA binding and nanoparticle formation, additional steps may be implemented in the above method at the same time.

The polypeptide nanoparticle produced by the foregoing method forms a nanoparticle from the polypeptide complex and various types of nucleic acid through self-assembly in aqueous solution. A chemotherapeutic drug can also be introduced into the composite to formulate into the nanoparticle for treating a specific disease, for example, cancer, scarring, and inflammatory disease. An example is the incorporation of gemcitabine or 5-FU or Cisplatin for treatment of cancer.

The size of the polypeptide nanoparticle in the present invention may range from 10 nm to 3000 nm based on the described production method. Depending on the preclinical study, the preferred size is 80-130 nm (as determined using a dynamic light scattering instrument to measure particle size and distribution).

In addition, the HKC2 polypeptide-nucleic acid delivery system according to the present invention may be used as an effective pharmaceutical composition. Therefore, the current invention provides a pharmaceutical composition comprising an effective dose of the HKC2 peptide and a nucleic acid. It may include one or more kinds of pharmaceutically compatible polymers or carriers in addition to the HKC2 polypeptide—nucleic acid delivery system for administration.

The resulting product can be formulated in various ways, such as in liquid, solid form, capsule, injectable, or the like with mixing of one or more effective ingredients such as saline solution, buffer solution, or other compatible ingredients to maintain the stability and effectiveness of the nucleic acid-peptide/polypeptide nanoparticle.

The structure of the HKC2 was characterized by HPLC and mass spectroscopy, and a major peak at retention time at 8.053 min with a purity 90.0% was observed by RPHPLC. In the ESI-MS spectrum (FIG. 2B), a molecular ion peak was observed as double charged ion [M+2H]²⁺. Similarly, triple charged, 4+ and 5+ species were also observed. It provided a molecular weight of 2683 Da, which is in good agreement with the theoretical value. The net charge on the peptide is 6+ at pH 7.0, so it is readily soluble in water (FIG. 2A). This is a plus for its formulation with the siRNA in aqueous medium.

### RNAi Therapeutic Approach

We have used a polypeptide-based carrier known as Histidine-Lysine Polymer (HKP), to deliver siRNAs in vitro and in vivo. This technology (see U.S. Pat. No. 8,735,567 B2, issued May 27, 2014 and U.S. Pat. No. 9,642,873 B2, issued May 9, 2017, which are incorporated herein by reference in their entireties) is able to substantially enhance delivery of siRNAs to the appropriate cells in the diseased tissue where they exert their effect to silence their targeted mRNA, blocking production of the protein and therefore impacting disease states e.g. scar healing, liver fibrosis disease, and cancer amongst others.

### RNAi and Therapeutic Agents

RNAi is a potent method that can be used to knock down gene expression, destroying an mRNA in a sequence-specific manner. RNAi can be managed to provide biological function in a rapid and sustained fashion. The present invention provides an RNAi delivery method for use in potential therapeutics. The invention provides many forms of siRNA molecules as therapeutic agents, including double stranded RNA (dsRNA) oligonucleotides (with or without overhang, sticky or blunt ends), small-hairpin RNA (shRNA), and DNA-derived RNA (ddRNA).

Design of siRNA Sequences

The RNAi agents are designed to have a nucleotide sequence matching a portion of the sequence of a targeted gene. The selected siRNA sequence of the targeted gene may be in any part of the mRNA generated by expression of the gene. The RNAi comprises a sequence that will hybridize with mRNA from the target gene—an “antisense strand” of the siRNA sequence. The siRNA sequence comprises a sequence that will hybridize with the antisense strand, a “sense strand” of the siRNA sequence. The siRNA sequence selected against the targeted gene should not be homologous with any other mRNA generated by the cell, nor with any sequence of the targeted gene that is not transcribed into mRNA. Numerous design rules for selecting a sequence of 20 to 27 bases of the target mRNA sequence are known, including commercially available methods. Designs can be obtained from at least three methods and a single consensus list of highest priority constructed and assembled from these methods. We have found that preparation of at least 6 of the highest priority candidate sequences, followed by cell culture testing for gene inhibition, nearly always reveals at least two active siRNA sequences. If not, a second round (obtaining six highest priority candidate sequences and testing) can be used.

Besides identification of active siRNA sequences, the design also must ensure homology only with the target mRNA sequences. A poor homology of siRNA sequences with genomic sequences other than those of the target gene mRNA reduces off-target effects at either the mRNA level or the gene level. Also, a poor homology of the “sense strand” of the siRNA sequence reduces off-target effects. By DNA comparison using Clone Manager Suite and by on-line Blast search, the targeted sequences of the selected gene can be confirmed to be unique and to lack sequence homology for other genes, including human counterparts. For example, sequences matching the mRNA of mVEGF-A are confirmed to be unique for mVEGF-A without homology for mVEGF-B mRNA, mVEGF-C mRNA, mVEGF-D mRNA, or human counterparts including hVEGF165-a (AF486837). However, the matching sequences will target multiple isoforms of mVEGF-A, e.g., mVEGF (M95200), mVEGF115 (U502791), mVEGF-2 (S38100), mVEGF-A (NM.sub.-192823), that encode mVEGF-A proteins of 190 amino acid (aa), 141 aa, 146 aa, and 148 aa, respectively. All of the published cDNA sequences of these mVEGF-A isofoms, except mVEGF-A (NM. sub.-192823, a mature form of protein), include a 26-aa signal peptide at the N-terminus. The targeted sequences of mVEGF are chosen not in the signal peptide part, but in the mature protein part shared by all these mVEGF-A isoforms.

Targeted sequences of mVEGF-R2 are also confirmed to be unique for these two genes, respectively. Different forms of interfering RNAs are included in the present invention. As an example, siRNA sequences are designed according to the above target sequences, using known guidelines. These siRNAs are 25 blunt end stranded RNA oligos (Table 1-3).

The RNAi agents are specific for the target gene sequence, which is dependent upon what species of the organism (animal) we are trying to target. Most mammalian genes share considerable homology, where RNAi agents can be selected to give activity for genes in multiple species with that homologous segment of mRNA of the gene of interest. The preferred siRNA inhibitor design should have perfect homology with both human gene mRNA and a test animal gene mRNA. The test animal(s) should be the one commonly used for efficacy and toxicity studies, such as mouse, rabbit or monkey.

Since it is known that a minimum of 17-nucleotides (nt) homologous to other gene sequences is required for an siRNA to generate sequence dependent off-target effects, a blast for each of the 8 possible 17 nt sequences from one 25-mer siRNA may be necessary to investigate the potential of sequence-dependent off-target effect, and use this information as one important parameter for finalizing the selection of siRNA for API (active pharmaceutical ingredient) of several siRNA therapeutic programs.

We also checked the siRNA candidates to exclude those containing the known immune stimulatory motif (GU-Rich region, 5′-UGUGU-3′ or 5′-GUCCUUCAA-3′) that may induce the activation of IFN pathway in vivo and in vitro via the TLRs pathway, although our RPP delivery system is highly unlikely to induce the TOLL-like receptor mediated activation of interferon pathway. Finally, we also mapped the targeting region of each tested siRNA sequence to their target mRNA sequences. This mapping is particularly useful for understanding the targeting capability of siRNA candidate on target mRNA and its alternative transcripts.

The selection of potent siRNA targeting sequences is listed in the tables below. SiRNA sequences selected were tested in the in vitro cell line first and followed by the in vivo testing for potency and efficacy by complexing with the selected transfection agent prior to administration.

TABLE 1
Selection of potent siRNA targeting VEGF:

| | | |
|---|---|---|
| hmVEGFa: | Sense: | 5′r(CCAUGCCAAGUGG |
| | | UCCCAGGCUGCA)-3′ |
| | | (SEQ ID NO: 10) |
| | Anti-sense: | 5′-r(UGCAGCCUGGG |
| | | ACCACUUGGCAUGG)-3′ |
| | | (SEQ ID NO: 11) |
| hmVEGFb: | Sense: | 5′-r(CCAACAUCACCA |
| | | UGCAGAUUAUGCG)-3′ |
| | | (SEQ ID NO: 12) |
| | Anti-sense: | 5′r(CGCAUAAUCUGCA |
| | | UGGUGAUGUUGG)-3′ |
| | | (SEQ ID NO: 13) |
| hmVEGFc: | Sense: | 5′r(CUGUAGACACACC |
| | | CACCCACAUACA)-3′ |
| | | (SEQ ID NO: 14) |
| | Anti-sense: | 5′-r(UGUAUGUGGGUG |
| | | GGUGUGUCUACAG)-3′ |
| | | (SEQ ID NO: 15) |
| hmVEGFd: | Sense: | 5′-r(CACUUUGGGUCC |
| | | GGAGGGCGAGACU)-3′ |
| | | (SEQ ID NO: 16) |
| | Anti-sense: | 5′r(AGUCUCGCCCUCC |
| | | GGACCCAAAGUG)-3′ |
| | | (SEQ ID NO: 17) |
| hmVEGFe: | Sense: | 5′r(CCUGAUGAGAUC |
| | | GAGUACAUCUUCA)-3′ |
| | | (SEQ ID NO: 18) |
| | Anti-sense: | 5′-r(UGAAGAUGUAC |
| | | UCGAUCUCAUCAGG)-3′ |
| | | (SEQ ID NO: 19) |
| hmVEGFf: | Sense: | 5′-r(GAGAGAUGAGC |
| | | UUCCUACAGCACAA)-3′ |
| | | (SEQ ID NO: 20) |
| | Anti-sense: | 5′r(UUGUGCUGUAGGA |
| | | AGCUCAUCUCUC)-3′ |
| | | (SEQ ID NO: 21) |
| hmVEGFg: | Sense: | 5′r(GCAAGGCGAGGCA |
| | | GCUUGAGUUAAA)-3′ |
| | | (SEQ ID NO: 22) |
| | Anti-sense: | 5′-r(UUUAACUCAAGC |
| | | UGCCUCGCCUUGC)-3′ |
| | | (SEQ ID NO: 23) |
| hmVEGFh: | Sense: | 5′-r(CACAACAAAUGU |
| | | GAAUGCAGACCAA)-3′ |
| | | (SEQ ID NO: 24) |
| | Anti-sense: | 5′r(UUGGUCUGCAUUC |
| | | ACAUUUGUUGUG)-3′ |
| | | (SEQ ID NO: 25) |

TABLE 2
Selection of potent siRNA targeting VEGFR2:

| | | |
|---|---|---|
| hVR2a: | Sense: | 5′r(CCUCUUCUGUAA |
| | | GACACUCACAAUU)-3′ |
| | | (SEQ ID NO: 26) |
| | Anti-sense: | 5′-r(AAUUGUGAGUGU |
| | | CUUACAGAAGAGG)-3′ |
| | | (SEQ ID NO: 27) |
| hVR2b: | Sense: | 5′-r(CCCUUGAGUCCAA |
| | | UCACACAAUUAA)-3′ |
| | | (SEQ ID NO: 28) |
| | Anti-sense: | 5′r(UUAAUUGUGUGAUU |
| | | GGACUCAAGGG)-3′ |
| | | (SEQ ID NO: 29) |
| hVR2c: | Sense: | 5′r(CCAAGUGAUUGAAG |
| | | CAGAUGCCUUU)-3′ |
| | | (SEQ ID NO: 30) |
| | Anti-sense: | 5′-r(AAAGGCAUCUGCU |
| | | UCAAUCACUUGG)-3′ |
| | | (SEQ ID NO: 31) |
| hmVR2d: | Sense: | 5′-r(GAGCAUGGAAGAG |
| | | GAUUCUGGACUC)-3′ |
| | | (SEQ ID NO: 32) |
| | Anti-sense: | 5′r(GAGUCCAGAAUCCU |
| | | CUUCCAUGCUC)-3′ |
| | | (SEQ ID NO: 33) |
| hmVR2e: | Sense: | 5′r(CAUGGAAGAGGAUU |
| | | CUGGACUCUCU)-3′ |
| | | (SEQ ID NO: 34) |
| | Anti-sense: | 5′-r(AGAGAGUCCAGAA |
| | | UCCUCUUCCAUG)-3′ |
| | | (SEQ ID NO: 35) |
| hmVR2f: | Sense: | 5′-r(CCUGACCUUGGAG |
| | | CAUCUCAUCUGU)-3′ |
| | | (SEQ ID NO: 36) |
| | Anti-sense: | 5′r(ACAGAUGAGAUGCU |
| | | CCAAGGUCAGG)-3′ |
| | | (SEQ ID NO: 37) |
| hmVR2g: | Sense: | 5′r(GCUAAGGGCAUGGA |
| | | GUUCUUGGCAU)-3′ |
| | | (SEQ ID NO: 38) |
| | Anti-sense: | 5′-r(AUGCCAAGAACUC |
| | | CAUGCCCUUAGC)-3′ |
| | | (SEQ ID NO: 39) |
| hmVR2h: | Sense: | 5′-r(GACUUCCUGACCU |
| | | UGGAGCAUCUCA)-3′ |
| | | (SEQ ID NO: 40) |
| | Anti-sense: | 5′r(UGAGAUGCUCCAAG |
| | | GUCAGGAAGUC)-3′ |
| | | (SEQ ID NO: 41) |

TABLE 3
Selection of potent siRNA targeting
TGF-Beta1:

| | | |
|---|---|---|
| hmTFβ1a: | Sense: | 5′r(GGAUCCACGAGCC |
| | | CAAGGGCUACCA)-3′ |
| | | (SEQ ID NO: 42) |
| | Anti-sense: | 5′-r(UGGUAGCCCUUG |
| | | GGCUCGUGGAUCC)-3′ |
| | | (SEQ ID NO: 43) |
| hmTFβ1b: | Sense: | 5′-r(CCCAAGGGCUAC |
| | | CAUGCCAACUUCU)-3′ |
| | | (SEQ ID NO: 44) |
| | Anti-sense: | 5′r(AGAAGUUGGCAUG |
| | | GUAGCCCUUGGG)-3′ |
| | | (SEQ ID NO: 45) |
| hmTFβ1c: | Sense: | 5′r(GAGCCCAAGGGCU |
| | | ACCAUGCCAACU)-3′ |
| | | (SEQ ID NO: 46) |
| | Anti-sense: | - 5′-r(AGUUGGCAUG |
| | | GUAGCCCUUGGG |
| | | CUC)-3′ |
| | | (SEQ ID NO: 47) |
| hmTFβ25d: | Sense: | 5′-r(GAUCCACGAGC |
| | Anti-sense: | CCAAGGGCUACCAU)-3′ |
| | | (SEQ ID NO: 48) |
| | | 5′r(AUGGUAGCCCUUG |
| | | GGCUCGUGGAUC)-3′ |
| | | (SEQ ID NO: 49) |
| hmTFβ25e: | Sense: | 5′r(CACGAGCCCAAGG |
| | Anti-sense: | GCUACCAUGCCA)-3′ |
| | | (SEQ ID NO: 50) |
| | | 5′-r(UGGCAUGGUAGC |
| | | CCUUGGGCUCGUG)-3′ |
| | | (SEQ ID NO: 51) |
| hmTFβ25f: | Sense: | 5′-r(GAGGUCACCCGC |
| | Anti-sense: | GUGCUAAUGGUGG)-3′ |
| | | (SEQ ID NO: 52) |
| | | 5′r(CCACCAUUAGCAC |
| | | GCGGGUGACCUC)-3′ |
| | | (SEQ ID NO: 53) |
| hmTFβ25g: | Sense: | 5′r(GUACAACAGCAC |
| | Anti-sense: | CCGCGACCGGGUG)-3′ |
| | | (SEQ ID NO: 54) |
| | | 5′-r(CACCCGGUCGC |
| | | GGGUGCUCUUCUAC)-3′ |
| | | (SEQ ID NO: 55) |
| hmTFβ25h: | Sense: | 5′-r(GUGGAUCCACGA |
| | Anti-sense: | GCCCAAGGGCUAC)-3′ |
| | | (SEQ ID NO: 56) |
| | | 5′r(GUAGCCCUUGGGC |
| | | UCGUGGAUCCAG)-3 |
| | | (SEQ ID NO: 57) |

As used herein, the singular forms “a,” “an,” and “the” refer to one or more, unless the context clearly indicates otherwise.

The following examples illustrate certain aspects of the invention and should not be construed as limiting the scope thereof.

### EXAMPLES

### Example 1. Cross-Linking of the Peptide Through Disulfide Bonds by Air

An initial study was conducted to examine the polypeptide formation through disulfide bond cross-linking of the peptide. The peptide HKC2 (3.0 mg) was dissolved in deionized water (0.5 mL) at room temperature, and the solution was stored at 4° C. for 10 hours. The resulting mixture was analyzed by reversed phase C-8 HPLC eluted by water (0.1% TFA) and acetonitrile (0.1% TFA), and it shows one peak on the chromatogram at a retention time of 3.3 min. There is no peak eluted at the retention time of 8.053 min representing the starting material-HKC2. It confirms that the peptide can be oxidized and cross-linked by air (FIG. 3).

### Example 2. Cross-Linking of the Peptide Through Disulfide Bonds by DMSO

The peptide HKC2 was similarly oxidized by the use of 5% DMSO in water. The peptide HKC2 (3.0 mg) was dissolved in deionized water at room temperature, and the solution was stored at 4° C. for 10 hours. The resulting mixture was analyzed by reversed phase C-8 HPLC eluted using water (0.1% TFA) and acetonitrile (0.1% TFA). It shows one peak on the chromatogram at a retention time of 3.3 min. There was no peak eluted at a retention time of 8.053 min for the starting material HKC2. It confirms that the peptide can be oxidized by DMSO (FIG. 3).

### Example 3. Nanoparticle Formation Through Self-Assembly Between Cross-Linked HKC2 Peptide and siRNA

After validating the cross linkage of HKC2 in water, we investigated the self-assembly between the HKC2 and siRNA (against TGF-β1). First, a concentrated stock solution of cross-linked HKC2 was prepared in water with 5% DMSO. A series of HKC2 in the various ratios with siRNA (wt:wt) (1:1, 2:1, 4:1, etc.) was mixed with siRNA and quickly stirred by vortexing. The size distribution of polypeptide nanoparticles between HKC2 and TGFβ1 measured by Dynamic Light Scattering instrumentation (DLS) was determined after 30 min. From the size distribution, under high concentration between the TGFβ1 (2.5 μg/μL) and HKC2 (30 μg/μL) in mixing ratio from 1:1 to 1:6, a higher nanoparticle size (2000^{˜}3000 nm) and precipitation was observed in some cases. The size remained the same no matter what addition sequence between siRNA and HKC2 was used (FIG. 1).

### Example 4. Intracellular Delivery of HKC2-siRNA PolyPeptide Nanoparticles (PNP) to HEK293 Cells

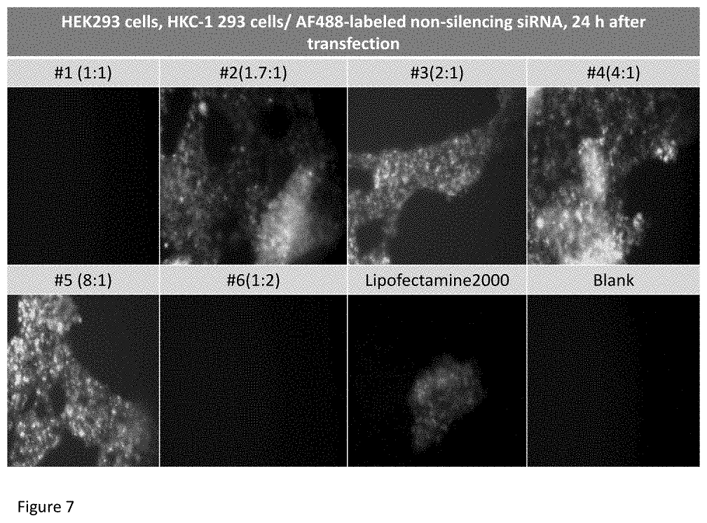

HEK293 cells were seeded at 3×10⁴ cells per well in 48-well plate and incubated overnight. On the next day AF488-labeled siRNA/HKC2 complexes were prepared as follows: Aqueous solutions of siRNA (0.025 μg/μL, 21-mer) and HKC2 (0.05 μg/μL) were combined at the following HKC2 to siRNA mass ratios: 1:1, 1.7:1, 2:1, 4:1, 8:1 and 1:2. After 30 min, siRNA/HKC2 complexes were added to the cells. Fluorescent images were taken 24 h after transfection. From the image in FIG. 7, we observed that siRNA was delivered inside of the cell (FIG. 7).

### Example 5. Intracellular Delivery of HKC2-siRNA PNP to A549 Cells

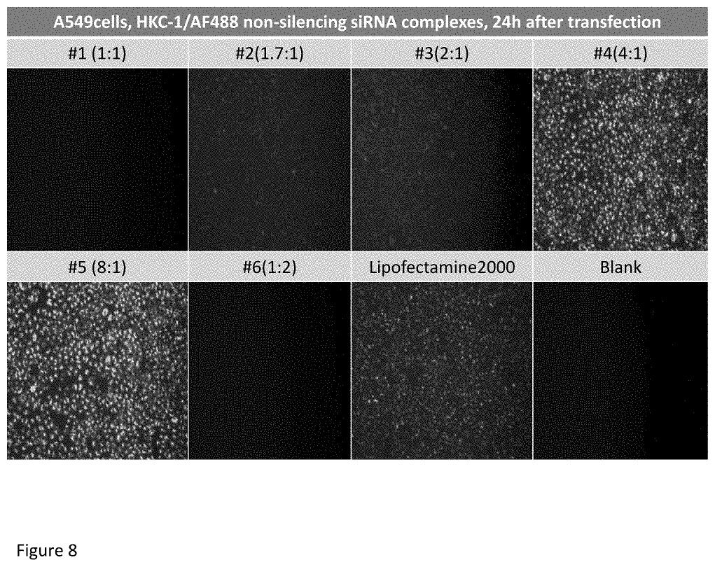

Fluorescently labeled siRNA (Alexa Fluor 488) in complex with HKC2 peptide was used to validate siRNA delivery. A549 cells were seeded in the wells of 48-well plate at a density of 3×10⁴ cells/well on the day before transfection. On the next day, AF488-labeled siRNA/HKC2 complexes were prepared as follows: Aqueous solutions of siRNA (0.025 μg/μL, 21-mer) and HKC2 (0.05 μg/μL) were combined at the following HKC2 to siRNA ratios: 1 to 1, 1.7 to 1, 2 to 1, 4 to 1, 8:1 and 1:2. After 30 min, siRNA/HKC2 complexes were added to the cells. Fluorescent images were taken 24 h after transfection. From the image in FIG. 8, we observed that siRNA was clearly delivered inside A549 cells (FIG. 8).

### Example 6. Gel Retardation Assay to Determine the Amount of HKC2 that Retards siRNA Migration

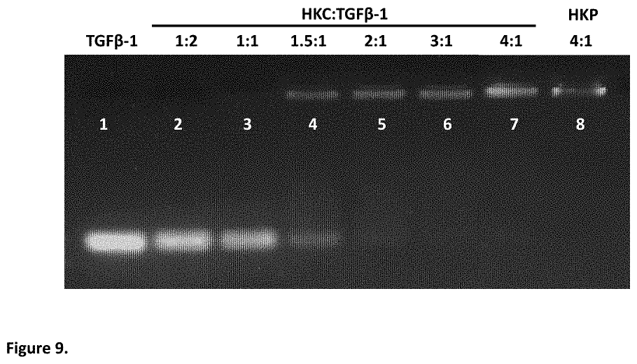

Various ratios of HKC2 in complex with siRNA (TGF-β1, 500 ng) were prepared and subjected to gel electrophoresis for 30 min (3% gel). Different ratios of HKC2 polypeptide to siRNA are represented above the gel (FIG. 9). In practice, 25 ng/μL of siRNA was incubated with various amounts of HKC2 peptide in ratio of 1:2, 1:1, 1.5:1, 2:1, 3:1, 4:1 or reference HKP (4:1). Following incubation for 20 min, 20 uL of siRNA/peptide (500 ng siRNA in each) complex was loaded into the wells within the gel. The free and bound siRNA were separated on a 3.0% non-denaturing agarose gel under 100V applied voltage for 30 min. The gel was stained with Ethituim bromide RNA dye, and the resulting fluorescent bands UV=290 nm were visualized with a Fuji LAS4000 Imager (FIG. 9).

### Example 7. Gel Retardation Assay to Validate the Degradation of HKC2 and Release of siRNA in the Presence of Glutathione (GSH)

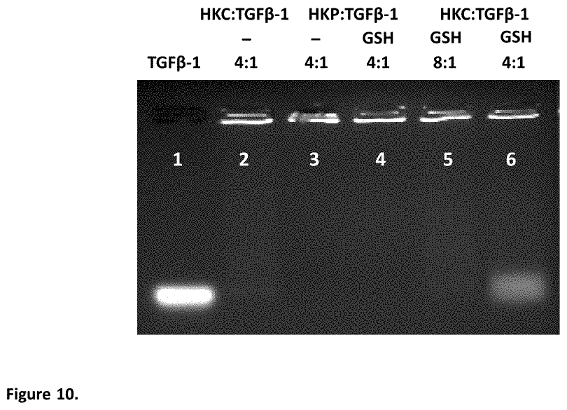

Various ratios of HKC2 or HKP in complex with siRNA (TGF-β1, 500 ng) were prepared and subjected to gel electrophoresis for 30 min (3% gel). Different ratios of HKC2 polypeptide to siRNA are represented above the gel (FIG. 10). In practice, 25 ng/μL of siRNA was incubated with various amounts of cross linked HKC2 peptide in ratio of 4:1 and 8:1. or reference HKP (4:1) in the presence or absence of 20 mM glutathione (GSH). Following the incubation for 40 min, 20 uL of siRNA/peptide (500 ng siRNA in each) complex was loaded in the wells of a gel. The free and bound siRNA was separated on a 3.0% agarose gel under 100V applied voltage for 30 min. The gel was stained with ethidium bromide, and the resulting fluorescent bands UV=290 nm was visualized with a Fuji LAS4000 Imager. The results presented are representative of the images obtained from multiple tests.

### Example 8. Size Distribution and Polydispersity of Formulation of HKC2:HKP:TGFβ1 in the Formation of Nanoparticle

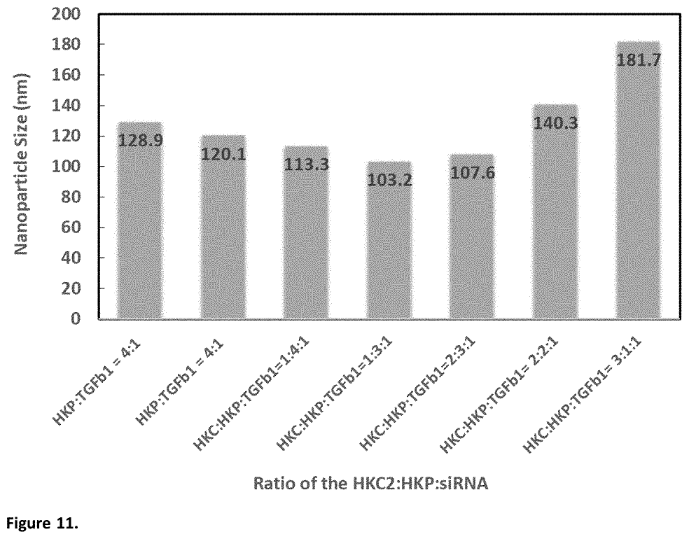

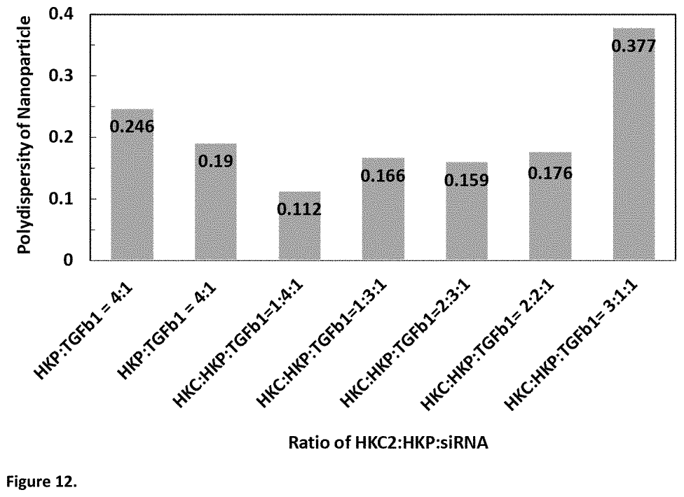

HKC2=K(HHHK)₄CSSC (SEQ ID NO: 3). HKP=H3K4b. TGFβ1 was used in 80 ng/μL in water. They were mixed with equal volume of the HKC and HKP in water. The nanoparticle formation of HKC2, HKP and siRNA (TGFβ1) was evaluated in various ratios. The addition of the HKC2 into the HKP/siRNA formulation maintained a similar nanoparticle size but significantly narrowed the polydispersity index (PDI) when compared to the control HKP/siRNA (N:P mass ratio=4:1). The HKC2/HKP/siRNA was formulated in mass ratio 0:4:1, 1:4:1, 1:3:1, 2:3:1, 2:2:1, 3:1:1. An aqueous solution of HKC2 (160 ng/μL), HKP (320 ng/μL) and siRNA (80 ng/μL) was mixed in the defined ratio and incubated at RT for 30 min. The resultant sample was subsequently measured by dynamic light scattering using a Nanoplus 90 instrument (Brookhaven). The dynamic radius and polydispersity were recorded and shown in FIGS. 11 and 12.

### Example 9. Effect of Treatment with Cell Death siRNA (Qiagen) Formulated with HKP Alone or in Combination with Various Amounts of HKP and HKC on Human Glioblastoma T98G Cell Line

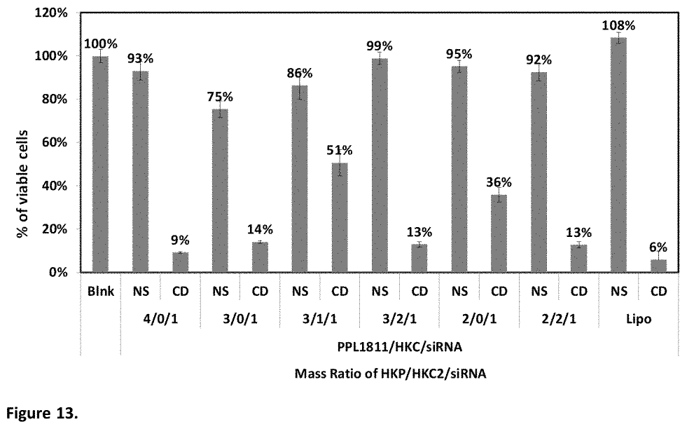

Various mass ratios of HKP/HKC2/siRNA were used and lipofectamine was also used as a control. At first an aqueous solution of HKC (160 ng/ul) was added to an aqueous solution of siRNA (80 ng/ul), mixed, briefly vortexed, then in the same manner HKP (320 ng/ul) was added. Mixtures were incubated at RT for 30 min. Transfection complexes were diluted with OPTI-MEM and added to the cells in 100 ul medium supplemented with fresh medium. 6 h after transfection, medium was replaced with 10% FBS/DMEM or EMEM. At 72 h post-transfection the number of viable cells was assessed with CellTiter-Glo Luminescent cell viability assay (Promega). Values derived from untreated cells (Blank) were set as 100%. All values represent the mean±S.D. of four replicates. NS-non-silencing siRNA (Qiagen, Germantown, Md.), CD-Cell Death siRNA (Qiagen, Germantown, Md.) (see FIG. 13).

### Example 10. Effect of Treatment with Cell Death siRNA (Qiagen) Formulated with HKP Alone or in Combination with Various Amounts of HKP and HKC on Human Hepatocellular Carcinoma HepG2 Cells

Various mass ratios of HKP/HKC2/siRNA were used and lipofectamine was used as a control. An aqueous solution of HKC (160 ng/ul) was added to an aqueous solution of siRNA (80 ng/ul), mixed, briefly vortexed, then HKP (320 ng/ul) was added. Mixtures were incubated at RT for 30 min. Transfection complexes were diluted with OPTI-MEM and added to the cells in 100 ul medium supplemented with fresh medium. 6 h after transfection, medium was replaced with 10% FBS/DMEM or EMEM. At 72 h post-transfection the number of viable cells was assessed with CellTiter-Glo Luminescent cell viability assay (Promega). Values derived from untreated cells (Blank) were set as 100%. All values represent the mean of ±S.D. of four replicates NS-non-silencing siRNA (Qiagen, Germantown, Md.), CD-CellDeath siRNA (Qiagen, Germantown, Md.).

### REFERENCES

1. Zimmermann T. S., Lee A. C., Akinc A., Bramlage B., Bumcrot D., Fedoruk M. N., MacKachlan I. (2006): RNAi-mediated gene silencing in non-human primates. Nature, 441, 111-114.
2. Judge A. D., Robbins M., Tabakoli L, Levi J., Hu L., Fronda A., Maclachian L. (2009): Confirming the RNAi-mediated mechanism of action of siRNA-based cancer therapeutics in mice. J. Clin, Invest., 119, 661-673.
3. Semple S. C., Akinc A., Chen J., Sandhu A. P., Mui B, L., Cho C. K., Hope M. J. (2010): Rational design of cationic lipids for siRNA delivery. Nat. Biotechnol., 28, 172-176.
4. Rozema D. B., Lewis D. L., Wakefield D. H., Wong S. C., Klein J. J., Roesch P. L., Bertin S. L., Reopen T. W., Chu Q., Blokhin A. V., Hagstrom J. E., Wolff 0.1. A. (2007): Dynamic PolyConjugates for targeted in vivo delivery of siRNA to hepatocytes. Proc. Natl. Acad. Sci. USA, 104, 12982-12987.
5. Wooddell C. I., Rozema D. B., Hossbach M., John M., Hamilton H. L., Chu Ct., Hegge J. O., Klein J. J., Wakefield D. H., Oropeza C. E., Decker J., Roehl I., Jahn-Hofmann K., Hadwiger P., Vornlocher H. P., McLachaln A., Lewis D. L. (2013): Hepatocyte-targeted RNAi therapeutics for the treatment of chronic hepatitis B virus infection. Mol. Ther., 21, 973-985.
6. Tatiparti K., Sau S., Kashaw S. K., Iyer A. K. (2017): siRNA Delivery Strategies: A Comprehensive review of recent developments, Nanomaterials (Basel). 7(4), e77.
7. U.S. Pat. No. 8,735,567 B2 of Lu et al., issued May 27, 2014 for Multi-Targeted RNAi Therapeutics for Starless Wound Healing of Skin.
8. U.S. Pat. No. 9,642,873 B2 of Lu et al., issued May 9, 2017 for Combinations of TGFβ and COX-2 Inhibitors and Methods for their Therapeutic Application.
9. Jia Zhou, Yixuan Zhao, Vera Simonenko, John J. Xu, Kai Liu, Deling Wang, Jingli Shi, Tianyi Zhong, Lixia Zhang, Lun Zeng, Bin Huang, Shenggao Tang, Alan Y. Lu, A. James Mixson, Yangbai Sun, Patrick Y. Lu and Qingfeng Li (2017): Simultaneous silencing of TGF-β1 and COX-2 reduces human skin hypertrophic scar through activation of fibroblast apoptosis, Oncotarget, 8, 80651-80665.
10. Cheng R., Feng F., Meng F., Deng C., Feijen J., Zhong Z. (2011): Glutathione-responsive nano-vehicles as a promising platform for targeted intracellular drug and gene delivery. J. Control. Release, 152, 2-12.
11. Zhu L., and Vladimir P. T., (2013): Stimulus-responsive nanopreparations for tumor targeting, Integr. Biol. (Camb). 5, 96-107.
12. Leng Q. and Mixson A. J. (2005): Modified branched peptides with a histidine-rich tail enhance in vitro gene transfection, Nucleic Acids Research, 33, e40.
13. Chou S. T., Horn K., Zhang D., Leng Q., Tricoli L. J., Hustedt J. M., Lee A., Shapiro M. J., Seog J., Kahn J. D., Mixson A. J. (2014): Enhanced silencing and stabilization of siRNA polyplexes by histidine-mediated hydrogen bonds, Biomaterials, 35, 846-855.
14. Anajafi T., Yu J., Sedigh A., Haldar M. K., Muhonen W. W., Oberlander S., Wasness H., Froberg J., Molla M. S., Katti K. S., Choi Y., Shabb J. B., Srivastava D. K., Mallik S. (2017): Nuclear Localizing Peptide-Conjugated, Redox-Sensitive Polymersomes for Delivering Curcumin and Doxorubicin to Pancreatic Cancer Microtumors, Mol. Pharmaceutics, 14, 1916-1928.
15. David P. F., Aline D. de A. (2016): Review Stapling Peptides Using Cysteine Crosslinking, PeptideScience, 106, 843-852.

The disclosures of all publications identified herein, including issued patents and published patent applications, and all database entries identified herein by url addresses or accession numbers are incorporated herein by reference in their entireties.

Although this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A peptide with the formula Kp{[(H)n(K)m]}y-C-x-Z (SEQ ID NO: 4 or the formula Kp{[(H)a(K)m(H)b(K)m (H)c(K)m(H)d(K)m]}y-C-x-Z (SEQ ID NO: 5), where K is lysine, H is histidine, C is cysteine, x is a linker, Z is a mammalian cell-targeting ligand, p is 0 or 1, n is an integer from 1 to 5, m is an integer from 0 to 3, a, b, c, and d are either 3 or 4, and y is an integer from 3 to 10.

2-11. (canceled)

12. A polypeptide comprising at least 2 of the peptides of claim 1 cross-linked through cleavable bonds.

13. (canceled)

14. The polypeptide of claim 12, wherein the cleavable bonds are disulfide bonds.

15-16. (canceled)

17. A nanoparticle comprising the polypeptide of claim 12 and a nucleic acid.

18. (canceled)

19. The nanoparticle of claim 17 further comprising a histidine-lysine copolymer.

20. (canceled)

21. The nanoparticle of claim 17, wherein the nucleic acid comprises an siRNA.

22-37. (canceled)

38. A method of delivering a nucleic acid to a mammalian cell comprising delivering the nanoparticle of claim 17 to the cell.

39-40. (canceled)

42. A method of gene therapy in a mammal comprising administering a therapeutically effective amount of the nanoparticles of claim 17 to the mammal.

43. The method of claim 42, wherein the mammal is a human.

44. A method of delivering a therapeutic compound to a mammal comprising delivering a therapeutically effective amount of the nanoparticles of claim 17 to the mammal.

45. The method of claim 44, wherein the mammal is a human.

46. A method of making the peptide of claim 11, comprising the steps of: a) linking the initial lysine (K) to a solid support; b) linking additional amino acids one after another to the initial lysine; and c) recovering the synthesized peptide.

47. A method of making a polypeptide, comprising the steps of: a) cross-linking the peptide of claim 1 by chemical oxidation to form a polypeptide with cleavable bonds, and b) recovering the polypeptide.

48. A method of making a nanoparticle, comprising the steps of: a) cross-linking the peptide of claim 1 by chemical oxidation to form a polypeptide with cleavable bonds, b) mixing the polypeptide with a nucleic acid, and c) recovering the nanoparticle.

49. A method of making a nanoparticle, comprising the steps of: a) mixing the polypeptide of claim 12 with a nucleic acid to form a nanoparticle, and b) recovering the nanoparticle.

50. A method of making a nanoparticle, comprising the steps of: a) mixing the peptide of claim 1 with a nucleic acid, b) cross-linking the peptide by chemical oxidation to form a polypeptide with cleavable bonds, resulting in the formation of a nanoparticle, and c) recovering the nanoparticle.

51-55. (canceled)

56. A peptide with the formula K[(H)n(K)m]y-C (SEQ ID NO: 7), where K is lysine, H is histidine, C is cysteine, n is an integer from 1 to 5, m is an integer from 0 to 3, y is an integer from 3 to 7.

57. (canceled)

58. A peptide with the formula K[(H)n(K)m]y-C-x-C (SEQ ID NO: 6, where K is lysine, H is histidine, C is cysteine, n is an integer from 1 to 5, m is an integer from 0 to 3, y is an integer from 3 to 7, and x is a linker.

59-62. (canceled)

63. A polypeptide comprising 2-10 of the peptides of claim 56 cross-linked through cleavable bonds.

64. (canceled)

65. A polypeptide comprising 2-10 of the peptides of claim 58 cross-linked through cleavable bonds.


SEQUENCE LISTING (59 sequences)

SEQ ID NO: 1 - protein, 18 aa, Artificial Sequence
  Description of Artificial Sequence Synthetic peptide
KHHHKHHHKHHHKHHHKC

SEQ ID NO: 2 - protein, 19 aa, Artificial Sequence
  Description of Artificial Sequence Synthetic peptide
KHHHKHHHHKHHHKHHHKC

SEQ ID NO: 3 - protein, 21 aa, Artificial Sequence
  Description of Artificial Sequence Synthetic peptide
KHHHKHHHKHHHKHHHKCSSC

SEQ ID NO: 4 - protein, 82 aa, Artificial Sequence
  Description of Artificial Sequence Synthetic polypeptide
  MISC_FEATURE (1)..(1): May or may not be present
  MISC_FEATURE (2)..(6): This region may encompass 1-5 residues
  MISC_FEATURE (7)..(9): This region may encompass 0-3 residues
  MISC_FEATURE (10)..(14): This region may encompass 1-5 residues
  MISC_FEATURE (15)..(17): This region may encompass 0-3 residues
  MISC_FEATURE (18)..(22): This region may encompass 1-5 residues
  MISC_FEATURE (23)..(25): This region may encompass 0-3 residues
  MISC_FEATURE (26)..(30): This region may encompass 1-5 residues
  MISC_FEATURE (31)..(33): This region may encompass 0-3 residues
  MISC_FEATURE (34)..(38): This region may encompass 1-5 residues
  MISC_FEATURE (39)..(41): This region may encompass 0-3 residues
  MISC_FEATURE (42)..(46): This region may encompass 1-5 residues
  MISC_FEATURE (47)..(49): This region may encompass 0-3 residues
  MISC_FEATURE (50)..(54): This region may encompass 1-5 residues
  MISC_FEATURE (55)..(57): This region may encompass 0-3 residues
  MISC_FEATURE (58)..(62): This region may encompass 1-5 residues
  MISC_FEATURE (63)..(65): This region may encompass 0-3 residues
  MISC_FEATURE (66)..(70): This region may encompass 1-5 residues
  MISC_FEATURE (71)..(73): This region may encompass 0-3 residues
  MISC_FEATURE (74)..(78): This region may encompass 1-5 residues
  MISC_FEATURE (79)..(81): This region may encompass 0-3 residues
  MISC_FEATURE (2)..(81): This region may encompass 3-10 “(H)n(K)m” units, wherein n = 1-5 and m = 0-3
KHHHHHKKKHHHHHKKKHHHHHKKKHHHHHKKKHHHHHKKKHHHHHKKKHHHHHKKKHHHHHKKKHHHHHKKKHHHHHKKKC

SEQ ID NO: 5 - protein, 282 aa, Artificial Sequence
  Description of Artificial Sequence Synthetic polypeptide
  MISC_FEATURE (1)..(1): May or may not be present
  MISC_FEATURE (2)..(5): This region may encompass 3 or 4 residues
  MISC_FEATURE (6)..(8): This region may encompass 0-3 residues
  MISC_FEATURE (9)..(12): This region may encompass 3 or 4 residues
  MISC_FEATURE (13)..(15): This region may encompass 0-3 residues
  MISC_FEATURE (16)..(19): This region may encompass 3 or 4 residues
  MISC_FEATURE (20)..(22): This region may encompass 0-3 residues
  MISC_FEATURE (23)..(26): This region may encompass 3 or 4 residues
  MISC_FEATURE (27)..(29): This region may encompass 0-3 residues
  MISC_FEATURE (30)..(33): This region may encompass 3 or 4 residues
  MISC_FEATURE (34)..(36): This region may encompass 0-3 residues
  MISC_FEATURE (37)..(40): This region may encompass 3 or 4 residues
  MISC_FEATURE (41)..(43): This region may encompass 0-3 residues
  MISC_FEATURE (44)..(47): This region may encompass 3 or 4 residues
  MISC_FEATURE (48)..(50): This region may encompass 0-3 residues
  MISC_FEATURE (51)..(54): This region may encompass 3 or 4 residues
  MISC_FEATURE (55)..(57): This region may encompass 0-3 residues
  MISC_FEATURE (58)..(61): This region may encompass 3 or 4 residues
  MISC_FEATURE (62)..(64): This region may encompass 0-3 residues
  MISC_FEATURE (65)..(68): This region may encompass 3 or 4 residues
  MISC_FEATURE (69)..(71): This region may encompass 0-3 residues
  MISC_FEATURE (72)..(75): This region may encompass 3 or 4 residues
  MISC_FEATURE (76)..(78): This region may encompass 0-3 residues
  MISC_FEATURE (79)..(82): This region may encompass 3 or 4 residues
  MISC_FEATURE (83)..(85): This region may encompass 0-3 residues
  MISC_FEATURE (86)..(89): This region may encompass 3 or 4 residues
  MISC_FEATURE (90)..(92): This region may encompass 0-3 residues
  MISC_FEATURE (93)..(96): This region may encompass 3 or 4 residues
  MISC_FEATURE (97)..(99): This region may encompass 0-3 residues
  MISC_FEATURE (100)..(103): This region may encompass 3 or 4 residues
  MISC_FEATURE (104)..(106): This region may encompass 0-3 residues
  MISC_FEATURE (107)..(110): This region may encompass 3 or 4 residues
  MISC_FEATURE (111)..(113): This region may encompass 0-3 residues
  MISC_FEATURE (114)..(117): This region may encompass 3 or 4 residues
  MISC_FEATURE (118)..(120): This region may encompass 0-3 residues
  MISC_FEATURE (121)..(124): This region may encompass 3 or 4 residues
  MISC_FEATURE (125)..(127): This region may encompass 0-3 residues
  MISC_FEATURE (128)..(131): This region may encompass 3 or 4 residues
  MISC_FEATURE (132)..(134): This region may encompass 0-3 residues
  MISC_FEATURE (135)..(138): This region may encompass 3 or 4 residues
  MISC_FEATURE (139)..(141): This region may encompass 0-3 residues
  MISC_FEATURE (142)..(145): This region may encompass 3 or 4 residues
  MISC_FEATURE (146)..(148): This region may encompass 0-3 residues
  MISC_FEATURE (149)..(152): This region may encompass 3 or 4 residues
  MISC_FEATURE (153)..(155): This region may encompass 0-3 residues
  MISC_FEATURE (156)..(159): This region may encompass 3 or 4 residues
  MISC_FEATURE (160)..(162): This region may encompass 0-3 residues
  MISC_FEATURE (163)..(166): This region may encompass 3 or 4 residues
  MISC_FEATURE (167)..(169): This region may encompass 0-3 residues
  MISC_FEATURE (170)..(173): This region may encompass 3 or 4 residues
  MISC_FEATURE (174)..(176): This region may encompass 0-3 residues
  MISC_FEATURE (177)..(180): This region may encompass 3 or 4 residues
  MISC_FEATURE (181)..(183): This region may encompass 0-3 residues
  MISC_FEATURE (184)..(187): This region may encompass 3 or 4 residues
  MISC_FEATURE (188)..(190): This region may encompass 0-3 residues
  MISC_FEATURE (191)..(194): This region may encompass 3 or 4 residues
  MISC_FEATURE (195)..(197): This region may encompass 0-3 residues
  MISC_FEATURE (198)..(201): This region may encompass 3 or 4 residues
  MISC_FEATURE (202)..(204): This region may encompass 0-3 residues
  MISC_FEATURE (205)..(208): This region may encompass 3 or 4 residues
  MISC_FEATURE (209)..(211): This region may encompass 0-3 residues
  MISC_FEATURE (212)..(215): This region may encompass 3 or 4 residues
  MISC_FEATURE (216)..(218): This region may encompass 0-3 residues
  MISC_FEATURE (219)..(222): This region may encompass 3 or 4 residues
  MISC_FEATURE (223)..(225): This region may encompass 0-3 residues
  MISC_FEATURE (226)..(229): This region may encompass 3 or 4 residues
  MISC_FEATURE (230)..(232): This region may encompass 0-3 residues
  MISC_FEATURE (233)..(236): This region may encompass 3 or 4 residues
  MISC_FEATURE (237)..(239): This region may encompass 0-3 residues
  MISC_FEATURE (240)..(243): This region may encompass 3 or 4 residues
  MISC_FEATURE (244)..(246): This region may encompass 0-3 residues
  MISC_FEATURE (247)..(250): This region may encompass 3 or 4 residues
  MISC_FEATURE (251)..(253): This region may encompass 0-3 residues
  MISC_FEATURE (254)..(257): This region may encompass 3 or 4 residues
  MISC_FEATURE (258)..(260): This region may encompass 0-3 residues
  MISC_FEATURE (261)..(264): This region may encompass 3 or 4 residues
  MISC_FEATURE (265)..(267): This region may encompass 0-3 residues
  MISC_FEATURE (268)..(271): This region may encompass 3 or 4 residues
  MISC_FEATURE (272)..(274): This region may encompass 0-3 residues
  MISC_FEATURE (275)..(278): This region may encompass 3 or 4 residues
  MISC_FEATURE (279)..(281): This region may encompass 0-3 residues
  MISC_FEATURE (2)..(281): This region may encompass 3-10 “(H)a(K)m(H)b(K) m(H)c(K)m(H)d (K)m” units, wherein a, b, c, and d = 3 or 4 and m = 0-3
KHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKHHHHKKKC

SEQ ID NO: 6 - protein, 58 aa, Artificial Sequence
  Description of Artificial Sequence Synthetic polypeptide
  MISC_FEATURE (2)..(6): This region may encompass 1-5 residues
  MISC_FEATURE (7)..(9): This region may encompass 0-3 residues
  MISC_FEATURE (10)..(14): This region may encompass 1-5 residues
  MISC_FEATURE (15)..(17): This region may encompass 0-3 residues
  MISC_FEATURE (18)..(22): This region may encompass 1-5 residues
  MISC_FEATURE (23)..(25): This region may encompass 0-3 residues
  MISC_FEATURE (26)..(30): This region may encompass 1-5 residues
  MISC_FEATURE (31)..(33): This region may encompass 0-3 residues
  MISC_FEATURE (34)..(38): This region may encompass 1-5 residues
  MISC_FEATURE (39)..(41): This region may encompass 0-3 residues
  MISC_FEATURE (42)..(46): This region may encompass 1-5 residues
  MISC_FEATURE (47)..(49): This region may encompass 0-3 residues
  MISC_FEATURE (50)..(54): This region may encompass 1-5 residues
  MISC_FEATURE (55)..(57): This region may encompass 0-3 residues
  MISC_FEATURE (2)..(57): This region may encompass 3-7 “(H)n(K)m” units, wherein n = 1-5 and m = 0-3
KHHHHHKKKHHHHHKKKHHHHHKKKHHHHHKKKHHHHHKKKHHHHHKKKHHHHHKKKC

SEQ ID NO: 7 - protein, 58 aa, Artificial Sequence; identical to SEQ ID NO: 6
  Description of Artificial Sequence Synthetic polypeptide
  MISC_FEATURE (2)..(6): This region may encompass 1-5 residues
  MISC_FEATURE (7)..(9): This region may encompass 0-3 residues
  MISC_FEATURE (10)..(14): This region may encompass 1-5 residues
  MISC_FEATURE (15)..(17): This region may encompass 0-3 residues
  MISC_FEATURE (18)..(22): This region may encompass 1-5 residues
  MISC_FEATURE (23)..(25): This region may encompass 0-3 residues
  MISC_FEATURE (26)..(30): This region may encompass 1-5 residues
  MISC_FEATURE (31)..(33): This region may encompass 0-3 residues
  MISC_FEATURE (34)..(38): This region may encompass 1-5 residues
  MISC_FEATURE (39)..(41): This region may encompass 0-3 residues
  MISC_FEATURE (42)..(46): This region may encompass 1-5 residues
  MISC_FEATURE (47)..(49): This region may encompass 0-3 residues
  MISC_FEATURE (50)..(54): This region may encompass 1-5 residues
  MISC_FEATURE (55)..(57): This region may encompass 0-3 residues
  MISC_FEATURE (2)..(57): This region may encompass 3-7 “(H)n(K)m” units, wherein n = 1-5 and m = 0-3

SEQ ID NO: 8 - protein, 4 aa, Artificial Sequence
  Description of Artificial Sequence Synthetic peptide
CSSC

SEQ ID NO: 9 - protein, 17 aa, Artificial Sequence
  Description of Artificial Sequence Synthetic peptide
KHHHKHHHKHHHKHHHK

SEQ ID NO: 10 - RNA, 25 nt, Artificial Sequence
  Description of Artificial Sequence Synthetic oligonucleotide
ccaugccaaguggucccaggcugca

SEQ ID NO: 11 - RNA, 25 nt, Artificial Sequence
  Description of Artificial Sequence Synthetic oligonucleotide
ugcagccugggaccacuuggcaugg

SEQ ID NO: 12 - RNA, 25 nt, Artificial Sequence
  Description of Artificial Sequence Synthetic oligonucleotide
ccaacaucaccaugcagauuaugcg

SEQ ID NO: 13 - RNA, 25 nt, Artificial Sequence
  Description of Artificial Sequence Synthetic oligonucleotide
cgcauaaucugcauggugauguugg

SEQ ID NO: 14 - RNA, 25 nt, Artificial Sequence
  Description of Artificial Sequence Synthetic oligonucleotide
cuguagacacacccacccacauaca

SEQ ID NO: 15 - RNA, 25 nt, Artificial Sequence
  Description of Artificial Sequence Synthetic oligonucleotide
uguaugugggugggugugucuacag

SEQ ID NO: 16 - RNA, 25 nt, Artificial Sequence
  Description of Artificial Sequence Synthetic oligonucleotide
cacuuuggguccggagggcgagacu

SEQ ID NO: 17 - RNA, 25 nt, Artificial Sequence
  Description of Artificial Sequence Synthetic oligonucleotide
agucucgcccuccggacccaaagug

SEQ ID NO: 18 - RNA, 25 nt, Artificial Sequence
  Description of Artificial Sequence Synthetic oligonucleotide
ccugaugagaucgaguacaucuuca

SEQ ID NO: 19 - RNA, 25 nt, Artificial Sequence
  Description of Artificial Sequence Synthetic oligonucleotide
ugaagauguacucgaucucaucagg

SEQ ID NO: 20 - RNA, 25 nt, Artificial Sequence
  Description of Artificial Sequence Synthetic oligonucleotide
gagagaugagcuuccuacagcacaa

SEQ ID NO: 21 - RNA, 25 nt, Artificial Sequence
  Description of Artificial Sequence Synthetic oligonucleotide
uugugcuguaggaagcucaucucuc

SEQ ID NO: 22 - RNA, 25 nt, Artificial Sequence
  Description of Artificial Sequence Synthetic oligonucleotide
gcaaggcgaggcagcuugaguuaaa

SEQ ID NO: 23 - RNA, 25 nt, Artificial Sequence
  Description of Artificial Sequence Synthetic oligonucleotide
uuuaacucaagcugccucgccuugc

SEQ ID NO: 24 - RNA, 25 nt, Artificial Sequence
  Description of Artificial Sequence Synthetic oligonucleotide
cacaacaaaugugaaugcagaccaa

SEQ ID NO: 25 - RNA, 25 nt, Artificial Sequence
  Description of Artificial Sequence Synthetic oligonucleotide
uuggucugcauucacauuuguugug

SEQ ID NO: 26 - RNA, 25 nt, Artificial Sequence
  Description of Artificial Sequence Synthetic oligonucleotide
ccucuucuguaagacacucacaauu

SEQ ID NO: 27 - RNA, 25 nt, Artificial Sequence
  Description of Artificial Sequence Synthetic oligonucleotide
aauugugagugucuuacagaagagg

SEQ ID NO: 28 - RNA, 25 nt, Artificial Sequence
  Description of Artificial Sequence Synthetic oligonucleotide
cccuugaguccaaucacacaauuaa

SEQ ID NO: 29 - RNA, 25 nt, Artificial Sequence
  Description of Artificial Sequence Synthetic oligonucleotide
uuaauugugugauuggacucaaggg

SEQ ID NO: 30 - RNA, 25 nt, Artificial Sequence
  Description of Artificial Sequence Synthetic oligonucleotide
ccaagugauugaagcagaugccuuu

SEQ ID NO: 31 - RNA, 25 nt, Artificial Sequence
  Description of Artificial Sequence Synthetic oligonucleotide
aaaggcaucugcuucaaucacuugg

SEQ ID NO: 32 - RNA, 25 nt, Artificial Sequence
  Description of Artificial Sequence Synthetic oligonucleotide
gagcauggaagaggauucuggacuc

SEQ ID NO: 33 - RNA, 25 nt, Artificial Sequence
  Description of Artificial Sequence Synthetic oligonucleotide
gaguccagaauccucuuccaugcuc

SEQ ID NO: 34 - RNA, 25 nt, Artificial Sequence
  Description of Artificial Sequence Synthetic oligonucleotide
cauggaagaggauucuggacucucu

SEQ ID NO: 35 - RNA, 25 nt, Artificial Sequence
  Description of Artificial Sequence Synthetic oligonucleotide
agagaguccagaauccucuuccaug

SEQ ID NO: 36 - RNA, 25 nt, Artificial Sequence
  Description of Artificial Sequence Synthetic oligonucleotide
ccugaccuuggagcaucucaucugu

SEQ ID NO: 37 - RNA, 25 nt, Artificial Sequence
  Description of Artificial Sequence Synthetic oligonucleotide
acagaugagaugcuccaaggucagg

SEQ ID NO: 38 - RNA, 25 nt, Artificial Sequence
  Description of Artificial Sequence Synthetic oligonucleotide
gcuaagggcauggaguucuuggcau

SEQ ID NO: 39 - RNA, 25 nt, Artificial Sequence
  Description of Artificial Sequence Synthetic oligonucleotide
augccaagaacuccaugcccuuagc

SEQ ID NO: 40 - RNA, 25 nt, Artificial Sequence
  Description of Artificial Sequence Synthetic oligonucleotide
gacuuccugaccuuggagcaucuca

SEQ ID NO: 41 - RNA, 25 nt, Artificial Sequence
  Description of Artificial Sequence Synthetic oligonucleotide
ugagaugcuccaaggucaggaaguc

SEQ ID NO: 42 - RNA, 25 nt, Artificial Sequence
  Description of Artificial Sequence Synthetic oligonucleotide
ggauccacgagcccaagggcuacca

SEQ ID NO: 43 - RNA, 25 nt, Artificial Sequence
  Description of Artificial Sequence Synthetic oligonucleotide
ugguagcccuugggcucguggaucc

SEQ ID NO: 44 - RNA, 25 nt, Artificial Sequence
  Description of Artificial Sequence Synthetic oligonucleotide
cccaagggcuaccaugccaacuucu

SEQ ID NO: 45 - RNA, 25 nt, Artificial Sequence
  Description of Artificial Sequence Synthetic oligonucleotide
agaaguuggcaugguagcccuuggg

SEQ ID NO: 46 - RNA, 25 nt, Artificial Sequence
  Description of Artificial Sequence Synthetic oligonucleotide
gagcccaagggcuaccaugccaacu

SEQ ID NO: 47 - RNA, 25 nt, Artificial Sequence
  Description of Artificial Sequence Synthetic oligonucleotide
aguuggcaugguagcccuugggcuc

SEQ ID NO: 48 - RNA, 25 nt, Artificial Sequence
  Description of Artificial Sequence Synthetic oligonucleotide
gauccacgagcccaagggcuaccau

SEQ ID NO: 49 - RNA, 25 nt, Artificial Sequence
  Description of Artificial Sequence Synthetic oligonucleotide
augguagcccuugggcucguggauc

SEQ ID NO: 50 - RNA, 25 nt, Artificial Sequence
  Description of Artificial Sequence Synthetic oligonucleotide
cacgagcccaagggcuaccaugcca

SEQ ID NO: 51 - RNA, 25 nt, Artificial Sequence
  Description of Artificial Sequence Synthetic oligonucleotide
uggcaugguagcccuugggcucgug

SEQ ID NO: 52 - RNA, 25 nt, Artificial Sequence
  Description of Artificial Sequence Synthetic oligonucleotide
gaggucacccgcgugcuaauggugg

SEQ ID NO: 53 - RNA, 25 nt, Artificial Sequence
  Description of Artificial Sequence Synthetic oligonucleotide
ccaccauuagcacgcgggugaccuc

SEQ ID NO: 54 - RNA, 25 nt, Artificial Sequence
  Description of Artificial Sequence Synthetic oligonucleotide
guacaacagcacccgcgaccgggug

SEQ ID NO: 55 - RNA, 25 nt, Artificial Sequence
  Description of Artificial Sequence Synthetic oligonucleotide
cacccggucgcgggugcucuucuac

SEQ ID NO: 56 - RNA, 25 nt, Artificial Sequence
  Description of Artificial Sequence Synthetic oligonucleotide
guggauccacgagcccaagggcuac

SEQ ID NO: 57 - RNA, 25 nt, Artificial Sequence
  Description of Artificial Sequence Synthetic oligonucleotide
guagcccuugggcucguggauccag

SEQ ID NO: 58 - protein, 4 aa, Artificial Sequence
  Description of Artificial Sequence Synthetic peptide
GSSG

SEQ ID NO: 59 - protein, 19 aa, Artificial Sequence
  Description of Artificial Sequence Synthetic peptide
  MISC_FEATURE (9)..(9): May or may not be present
KHHHKHHHHKHHHKHHHKC